SEQUENCE LISTING (13 sequences)

SEQ ID NO: 1 - DNA, 50 bp, synthetic construct
  source 1..50: mol_type = other DNA
agcgaacgtggattttgtccgacatcggcaagctccctttttcgactatt

SEQ ID NO: 2 - DNA, 50 bp, synthetic construct
  source 1..50: mol_type = other DNA
ccgatgtcggacttttacacgatcttcgcctgctgggttttgggagcttg

SEQ ID NO: 3 - DNA, 50 bp, synthetic construct
  source 1..50: mol_type = other DNA
cgaagatcgtgtttttccacagttgattgcccttcacttttcccagcagg

SEQ ID NO: 4 - DNA, 50 bp, synthetic construct
  source 1..50: mol_type = other DNA
aatcaactgtggtttttctcactggtgattagaatgcttttgtgaagggc

SEQ ID NO: 5 - DNA, 50 bp, synthetic construct
  source 1..50: mol_type = other DNA
tcaccagtgagatttttgtcgtaccaggtgcatggatttttgcattctaa

SEQ ID NO: 6 - DNA, 50 bp, synthetic construct
  misc_structure 50: note = 3'-Alexa Fluor 488 attached to A50
  source 1..50: mol_type = other DNA
cctggtacgacatttttccacgttcgctaatagtcgattttatccatgca

SEQ ID NO: 7 - protein, 10 aa, synthetic construct
KKKKKKKKKK

SEQ ID NO: 8 - protein, 10 aa, synthetic construct
  MOD_RES 1: note = N-terminal Fluorescein attached to K1
KKKKKKKKKK

SEQ ID NO: 9 - protein, 11 aa, synthetic construct
  MOD_RES 11: note = C-terminal pHrodo Red attached to C11
KKKKKKKKKKC

SEQ ID NO: 10 - protein, 41 aa, synthetic construct
GLFDIIKKIAESFGSGKKKKKKKKKKGSGFEAIKKIIDFLG

SEQ ID NO: 11 - protein, 42 aa, synthetic construct
IKAFKGFDESILIGSGKKKKKKKKKKGSGILISEDFGKFAKI

SEQ ID NO: 12 - protein, 13 aa, synthetic construct
GLFDIIKKIAESF

SEQ ID NO: 13 - protein, 36 aa, synthetic construct
GLFDIIKKIAESFKKKKKKKKKKGLFDIIKKIAESF

(19) United States
(12) Patent Application Publication
(10) Pub. No.: US 2026/0273093 A1
(43) Pub. Date: Sep. 17, 2026

(54) CELLULAR UPTAKE OF FUNCTIONALIZED DNA NANOSTRUCTURES

(71) Applicant: ARIZONA BOARD OF REGENTS ON BEHALF OF ARIZONA STATE UNIVERSITY, Scottsdale, AZ (US)

(72) Inventor: Nicholas STEPHANOPOULOS, Scottsdale, AZ (US)

(21) Appl. No.: 19/690,748

(22) Filed: May 28, 2026

Related U.S. Application Data
(63) Continuation of application No. 18/063,824, filed on Dec. 9, 2022.
(60) Provisional application No. 63/287,838, filed on Dec. 9, 2021.

(51) Int. Cl. A61K 47/69; A61K 47/62; B82Y 5/00

(52) U.S. Cl. CPC A61K 47/6929; A61K 47/62; B82Y 5/00

(57) ABSTRACT

Described herein are DNA nanostructures (DN) functionalized with proteins and methods for cellular uptake. Cellular uptake of such DNs is linearly dependent on the cell size. The protein corona determines the endolysosomal vesicle escape efficiency of DNs coated with an endosome escape peptide.

### CROSS-REFERENCE TO RELATED APPLICATIONS

This application is a continuation of U.S. patent application Ser. No. 18/063,824, filed Dec. 9, 2022, which claims priority to U.S. Provisional Patent Application No. 63/287,838, filed on Dec. 9, 2021, each of which is incorporated by reference herein in its entirety.

### FEDERALLY SPONSORED RESEARCH

This invention was made with government support under grant number GM132931 awarded by the National Institutes of Health. The government has certain rights in the invention.

### REFERENCE TO SEQUENCE LISTING

This application was filed with a Sequence Listing XML in ST.26 XML format accordance with 37 C.F.R. § 1.831. The Sequence Listing XML file submitted in the USPTO Patent Center, “208192-9114-US03_sequence_listing_XML_20 May 2026.xml,” was created on May 20, 2026, contains 13 sequences, has a file size of 13.6 kilobytes (13,965 bytes), and is incorporated by reference in its entirety into the specification.

### TECHNICAL FIELD

Described herein are DNA nanostructures (DN) functionalized with proteins and methods for cellular uptake. Cellular uptake of such DNs is linearly dependent on the cell size. The protein corona determines the endolysosomal vesicle escape efficiency of DNs coated with an endosome escape peptide.

### BACKGROUND

In recent decades, nanoparticle (NP) vehicles have shown substantial potential in different biomedical applications, including the potential to change the biodistribution and pharmacokinetics of conventional free therapeutics. As a result, a plethora of NP formulations have been designed for uses like targeted drug delivery, imaging, biosensing, and other various biomedical and therapeutic applications. NPs designed as delivery vehicles were expected to solve several key persistent problems (e.g., degradation, poor solubility, toxicity, and incapability to cross biological barriers) of free drugs. Different formulations of NPs showed success in preclinical settings and clinical trials, and some NPs received clearance for clinical use. However, the majority of NP formulations possess very low success rates of clinical translation.

Despite isolated success cases, NP formulations are unable to reach maximal targeting effectiveness while concomitantly minimizing off-target effects. Poor knowledge of the fundamental cellular mechanisms of NP-cell interactions substantially contributes to such shortcomings. To a large extent, the capabilities to accurately produce NPs with tightly controlled size, shape, and surface chemistry are still rather limited. This in turn challenges the systematic investigation of NP-cell interactions, resulting in poor delivery efficacy.

DNA-based structural nanotechnology including polyhedral cages, bundles, or the complex assemblies afforded by DNA origami offer unique opportunities to build oligonucleotide nanostructures with tightly controlled size, shape, and surface functionality. Such remarkable molecular control over DNA nanostructures (DNs) has enabled applications like nanofabrication, biosensing, vehicles for spatiotemporal release of active compounds, cell engineering, and drug delivery. Importantly, DNs have been recognized as an alternative to conventional NP-based cargos for cellular delivery of various content, including small molecule drugs, proteins, and nucleic acids. Foreseeing potential clinical translation of DN-based applications, it is imperative to understand interactions between DNs and living cells in a well-defined and controlled manner. In fact, studies that analyze DN-cell interactions, as well as the ingestion routes and mechanisms of designed DNs are quite limited. For example, recent advancements were achieved in analyzing how the size and shape of DNs affect their cellular uptake. Although these reports analyze how different cell lines internalize DNs, no systematic investigation has been undertaken to directly compare the observed effects on closely related cell lines. Furthermore, from the nanoparticle field it is well established that, upon interaction with biological fluids, NPs form a so-called protein corona. Importantly, this protein corona affects the physicochemical characteristics of NPs but most importantly may change the overall bioreactivity of the nanoparticles. To current knowledge, there are no studies assessing the impact of this protein corona on the biological properties and delivery efficiency of functionalized DNs. Of note, such studies may open critical insights for the design and optimization of DNs for their successful clinical translation.

Thus, the cellular uptake and fate of functionalized DNs was studied in three closely related cell lines: HepG2, Huh7, and Alexander cells. A comparative analysis of DN uptake was performed in those cell lines and how the presence of serum proteins affects the desired bioreactivity of functionalized DNs. The effect of functionalizing the DNs with electrostatic peptide coatings was explored with an endosome escape peptide sequence for improving cytosolic delivery of the structures.

### SUMMARY

One embodiment described herein is a nanoparticle composition comprising a DNA nanostructure (DN) functionalized with an endolysosomal escape peptide. In one aspect, the DN comprises a 6-helix bundle (6HB) nanostructure. In another aspect, the 6HB nanostructure comprises six different double-stranded DNA helices, each DNA helix having a nucleotide sequence having at least 90-99% identity to SEQ ID NO: 1-6. In another aspect, the 6HB nanostructure comprises six different double-stranded DNA helices, each DNA helix having a nucleotide sequence selected from SEQ ID NO: 1-6. In another aspect, the 6HB nanostructure is a rigid and monomeric assembly roughly 7×6 nm² in size. In another aspect, the endolysosomal escape peptide coating comprises one or more endolysosomal escape peptides having an amino acid sequence having at least 90-95% identity to SEQ ID NO: 7-12. In another aspect, the endolysosomal escape peptide coating comprises one or more endolysosomal escape peptides having an amino acid sequence of SEQ ID NO: 7-12. In another aspect, the endolysosomal escape peptide comprises a lysine10 (K10) peptide (SEQ ID NO: 7). In another aspect, the endolysosomal escape peptide comprises an aurein 1.2 peptide (SEQ ID NO: 12). In another aspect, the endolysosomal escape peptide comprises a lysine10 (K10) peptide flanked by two copies of an aurein 1.2 peptide (SEQ ID NO: 10). In another aspect, the number of copies of the aurein 1.2 peptide per DN is equal to about 40 to about 50. In another aspect, the diameter of the functionalized DN is from about 15 nm to about 28 nm. In another aspect, the endolysosomal escape peptide coating binds the DN through electrostatic interactions at a nitrogen/phosphate ratio of about 0.8 to about 1.5. In another aspect, the nitrogen/phosphate ratio is about 1. In another aspect, the composition is stable in intracellular lysosomal compartments for up to 24 hr of incubation. In another aspect, the composition further comprises a therapeutic agent.

Another embodiment described herein is a method for improving cellular uptake of a DNA nanostructure (DN) through enhanced endolysosomal escape, the method comprising delivering to a cell a nanoparticle composition comprising a DN functionalized with an endolysosomal escape peptide coating. In one aspect, endolysosomal escape efficiency is determined by a protein corona. In another aspect, cellular uptake efficiency of the functionalized DN is linearly dependent on the cell size. In another aspect, the cell is a hepatoblastoma cell or a hepatocellular carcinoma cell. In another aspect, the endolysosomal escape peptide coating facilitates enhanced endolysosomal escape without concomitant disruption of a cell membrane and without cytotoxicity to the cell. In another aspect, the composition further comprises a therapeutic agent and the method is used to deliver the therapeutic agent to a cell.

### DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows a schematic overview of endolysosomal escape upon cellular uptake of functionalized DNs compared to naked proteins.

FIG. 2A-C show the design and functionalization of DNA nanostructures. FIG. 2A shows a strand diagram showing the six oligonucleotides that comprise the six-helix bundle (6HB) DN. Each oligonucleotide is marked with an arrow (5′->3′). Shown from left to right in the figure are SEQ ID NO: 6 (the first portion, which continues on the right side of the diagram following the dotted line, and the third portion, which is continued from the right side of the diagram following the dotted line), SEQ ID NO: 5, SEQ ID NO: 4, SEQ ID NO: 3, SEQ ID NO: 2, SEQ ID NO: 1, and the second portion of SEQ ID NO: 6, which is continued from the left side of the diagram following the dotted line. The linking thymidines for each sequence are shown at the termini and along the dotted lines for SEQ ID NO: 6. The 3′-terminal Alexa Fluor 488 is omitted from SEQ ID NO: 6 for simplicity. FIG. 2B shows schematics of the two peptides used for coating DNs in this study: a decalysine (“K10”) peptide (SEQ ID NO: 7) and K10 (SEQ ID NO: 7) flanked by two copies of the aurein 1.2 (SEQ ID NO: 12) (endolysosomal escape (SEQ ID NO: 13; “EE”) peptide. FIG. 2C shows schematics of the DNs coated with either the K10 peptide (SEQ ID NO: 7) (left) or an 80:20 mixture of EE/K10 (SEQ ID NO: 10/7) (right).

FIG. 3 shows a topology diagram showing the sequence and connectivity of the 6 strands (each shaded differently) that make up the 6-helix bundle DNA nanostructure. Each strand is marked with an arrow (5′->3′). Shown from left to right in the figure are SEQ ID NO: 6 (the first portion, which continues on the right side of the diagram following the dotted line, and the third portion, which is continued from the right side of the diagram following the dotted line), SEQ ID NO: 5, SEQ ID NO: 4, SEQ ID NO: 3, SEQ ID NO: 2, SEQ ID NO: 1, and the second portion of SEQ ID NO: 6, which is continued from the left side of the diagram following the dotted line. The linking thymidines for each sequence are shown at the termini and along the dotted lines for SEQ ID NO: 6. The 3′-terminal Alexa Fluor 488 is omitted from SEQ ID NO: 6 for simplicity. Each strand has four thymine in the linking regions between the duplexes.

FIG. 4A-E show MALDI-TOF mass spectra of the indicated purified peptides. All mass values are given in Da. FIG. 4A shows a plain K10 (SEQ ID NO: 7) with an observed mass of 1300.4 and an expected mass of 1298.8. FIG. 4B shows K10-fluorescein (SEQ ID NO: 8) with an observed mass of 1657.1 and an expected mass of 1659.5. FIG. 4C shows pHrodo labeled K10 (SEQ ID NO: 9) with an observed mass of 2089.2 and an expected mass of approximately 2108.9. FIG. 4D shows EE-K10 (SEQ ID NO: 10) with an observed mass of 2619.4 and an expected mass of 4626.7. FIG. 4E shows EE-K10 scramble (SEQ ID NO: 11) with an observed mass of 2617.6 and an expected mass of 4626.7.

FIG. 5A-B show agarose gel electrophoresis (1.5% agarose) used to determine the integrity of the 6-helix bundle as well as the optimal nitrogen to phosphate (N:P) ratio in order to fully coat (via electrostatic neutralization) the DNA nanostructure. FIG. 5B shows optimization of the N:P ratio around 0.8-1.5. For all experiments, the ratio of N:P=1 was used; below this value the DNs did not show a complete gel shift and increasing the peptide coating beyond this level yielded aggregation.

FIG. 6A-B show analyses of the stability of DNA nanostructures. (a) AFM characterization of the 6HB DN. (b) Overlay of the normalized FRET efficiency plots corresponding to cooling (dark) and heating cycles (grey) that reveals the reversible assembly and disassembly of the structure. 6HB was incubated for either 0 or 48 h in phosphate buffered saline (PBS), followed by determining the temperature of folding (T_{f}) and the temperature of melting (Tₘ) via melting profile analysis. Values are representative from three independent repeats.

FIG. 7A shows size distribution of different DNs. Characterization of the particles dissolved in PBS measured with a Zetasizer Nano (Malvern Instruments). FIG. 7B shows AFM characterization of the K10 and EE DNs.

FIG. 8 shows FRET-based monitoring of DNA nanostructure stability. Schematic representation of the 6HB structure labelled with FRET reporter dyes. Two selected staples are modified with FRET donor (6-carboxyfluorescein, circle labeled D) and acceptor (TAMRA, circle labeled A) dyes, respectively. Each strand is marked with an arrow (5′->3′). Shown from left to right in the figure are SEQ ID NO: 6 (the first portion, which continues on the right side of the diagram following the dotted line, and the third portion, which is continued from the right side of the diagram following the dotted line), SEQ ID NO: 5, SEQ ID NO: 4, SEQ ID NO: 3, SEQ ID NO: 2, SEQ ID NO: 1, and the second portion SEQ ID NO: 6, which is continued from the left side of the diagram following the dotted line. The linking thymidines for each sequence are shown at the termini and along the dotted lines for SEQ ID NO: 6. The FRET reporter dyes are omitted from the strand diagram for simplicity.

FIG. 9A-C show FRET-based monitoring of DNA nanostructure stability of freshly prepared 6HB. FIG. 9A shows raw fluorescent intensity versus temperature for the cooling cycle. FIG. 9B shows raw fluorescent intensity versus temperature for the heating cycle. FIG. 9C shows the derivative of the cooling curve and corresponding Gaussian fit to yield the transition temperature of folding, T_{f}. FIG. 9D shows the derivative of the heating curve and corresponding Gaussian fit to yield the transition temperature of melting, Tₘ.

FIG. 10A-D show FRET-based monitoring of DNA nanostructure stability of 6HB incubated at room temperature for 2 days. FIG. 10A shows raw fluorescent intensity versus temperature for the cooling cycle. FIG. 10B shows raw fluorescent intensity versus temperature for the heating cycle. FIG. 10C shows the derivative of the cooling curve and corresponding Gaussian fit to yield the transition temperature of folding, T_{f}. FIG. 10D shows the derivative of the heating curve and corresponding Gaussian fit to yield the transition temperature of melting, Tₘ.

FIG. 11A-C shows assessment of DNs biocompatibility in three distinct hepatic cell lines. FIG. 11A shows cytotoxicity of nanoparticles in three distinct cell lines: Alexander, HepG2, Huh7. Cells were treated with different DNs (10, 100 and 500 nM) for 24 h. Cytotoxicity was assessed using alamarBlue assay. The data were normalized to control values (no particle exposure), which were set as 100% cell viability. Control cells were untreated. As a positive control, cells were treated with 20% ethanol for 60 min. Data are expressed as means±SEM (n=3). FIG. 11B shows cells were treated with different DNs (500 nM) for 24 h. After treatment DIC images were acquired using inverted microscope IX83 (Olympus, Tokyo, Japan). As a positive control, cells were treated with 20% ethanol for 15 min. FIG. 11C shows cells were treated with different DNs for 24 h. After treatment, cells were labelled with CellMask™ Orange (Thermo Fisher Scientific) plasma membrane stain. Stained cells were imaged using spinning disk confocal microscopy IXplore SpinSR (Olympus, Tokyo, Japan). Representative images out of three independent experiments are presented. As a positive control, cells were treated with 20% ethanol for 15 min.

FIG. 12A-C show uptake of different DNs by three distinct hepatic cell lines. FIG. 12A shows Alexander, HepG2, and Huh7 cell lines were treated with a 50 nM concentration of different fluorescently labeled (green fluorescence) DNs for 24 h. After treatment, cells were stained using CellMask Orange (Thermo Fisher Scientific) plasma membrane stain. Stained cells were imaged using spinning disk confocal microscopy IXplore SpinSR (Olympus, Tokyo, Japan). 3D rendering orthogonal projections were done using ImageJ software (NIH). Representative images from three independent experiments are presented. White arrows indicate internalized DNs; grey arrows show DNs attached to the membrane surface; and green arrows depict extracellular DNs. FIG. 12B shows quantification of DNs uptake. Cells were treated and imaged as in FIG. 12A. The intracellular DNs were measured as corrected total cell fluorescence (CTCF) of the full area of interest using ImageJ software (NIH). Data are expressed out of at least three independent experiments (n=30 cells). (**) P<0.01 and (***) P<0.001 denote significant differences. FIG. 12C shows assessment of cell size and morphology in Huh7, HepG2, and Alexander cells. Cells were stained with CellMask Green (Thermo Fisher Scientific) plasma membrane stain. Nuclei were counterstained with Hoechst 33342 (Thermo Fisher Scientific). Stained cells were imaged using spinning disk confocal microscopy IXplore SpinSR (Olympus, Tokyo, Japan). Representative images out of three independent experiments are presented. 3D rendering orthogonal projections were done using ImageJ software (NIH).

FIG. 13A-B show uptake of different DNs by three distinct hepatic cell lines. FIG. 13A shows assessment of cell size and morphology in Huh7, HepG2 and Alexander cells. Cells were stained with CellMask™ Green (Thermo Fisher Scientific) plasma membrane stain. Nuclei were counterstained with Hoechst 33342 (Thermo Fisher Scientific). Stained cells were imaged using spinning disk confocal microscopy IXplore SpinSR (Olympus, Tokyo, Japan). Representative images out of three independent experiments are presented. FIG. 13B shows Alexander, HepG2, Huh7 cell lines were treated with 50 nM concentration of different fluorescently-labeled (green fluorescence) DNs for 24 h. After treatment, cells were fixed with 4% Paraformaldehyde (VWR) and labelled with CellBrite™ Blue (Biotium) plasma membrane stain. Stained cells were imaged using spinning disk confocal microscopy IXplore SpinSR (Olympus, Tokyo, Japan). 3D rendering orthogonal projections were done using ImageJ software (NIH). Representative images out of three independent experiments are presented.

FIG. 14 shows assessment of DNs uptake in three distinct hepatic cell lines. Alexander, HepG2, Huh7 cell lines were treated with 50 nM concentration of different DNs for 24 h. After treatment, cells were labelled with CellBrite™ Blue (Biotium) plasma membrane stain. Stained cells were imaged using spinning disk confocal microscopy IXplore SpinSR (Olympus, Tokyo, Japan). Representative images out of three independent experiments are presented.

FIG. 15A-D show uptake kinetics assessment of different DNs. FIG. 15A shows Alexander, HepG2, and Huh7 cell lines were treated with a 50 nM concentration of different DNs for 1, 6, and 24 h. After treatment, cells were fixed with 4% paraformaldehyde (VWR) and labeled with CellBrite Blue (Biotium) plasma membrane stain. Stained cells were imaged using spinning disk confocal microscopy IXplore SpinSR (Olympus, Tokyo, Japan). The intracellular DNs were measured as corrected total cell fluorescence (CTCF) of the full area of interest using ImageJ software (NIH). Data are expressed out of at least three independent experiments (n=28-34 cells). FIG. 15B shows assessment of cell size in Huh7, HepG2, and Alexander cells. Cells were stained with CellMask Green (Thermo Fisher Scientific) plasma membrane stain. Nuclei were counterstained with Hoechst 33342 (Thermo Fisher Scientific). Stained cells were imaged using spinning disk confocal microscopy IXplore SpinSR (Olympus, Tokyo, Japan). The average cell area was measured using ImageJ software (NIH) and is presented as means of n=30 cells. (***) P<0.001 denotes significant differences. FIG. 15C shows cell-size-dependent DNs uptake. The intracellular DNs presented as CTCF after 24 h treatment with 50 nM concentration of different DNs were plotted versus corresponding cell size. FIG. 15D shows linear correlation between cell size and DNs uptake. Each black point represents confocal microscopy-measured single-cell DN uptake plotted against corresponding cell size. The uptake is expressed as CTCF after 24 h treatment with 50 nM concentration of different DNs. Correlation coefficients and P values were calculated using SigmaPlot 13 software (Systat Software, Inc.).

FIG. 16 shows assessment of DNs uptake kinetics in three distinct hepatic cell lines. Alexander, HepG2, Huh7 cell lines were treated with 50 nM concentration of different DNs for 1, 6 and 24 h. After treatment, cells were labelled with CellBrite™ Blue (Biotium) plasma membrane stain. Stained cells were imaged using spinning disk confocal microscopy IXplore SpinSR (Olympus, Tokyo, Japan). Representative images of three independent experiments are shown.

FIG. 17 shows colocalization assessment of DNs in three distinct hepatic cell lines. Alexander, HepG2, Huh7 cell lines were treated with 50 nM concentration of different DNs for 6 h either in full medium (10% FBS EMEM) or in serum-free medium (0% FBS EMEM). After incubation cells were labelled with lysosomal marker LysoTracker® Blue DND-22 (Thermo Fisher Scientific). Stained cells were imaged using spinning disk confocal microscopy IXplore SpinSR (Olympus, Tokyo, Japan). Representative images of three independent experiments are shown. K10 and EE DNs have pHrodo (red) dye. DNA of all DNs was labelled with Alexa-488.

FIG. 18 shows FRET microscopy images of Alexander, HepG2, and Huh7 cells treated with 6HB labeled with FRET reporter dyes (6-carboxyfluorescein donor and TAMRA acceptor). Images of the three detection channels (donor, acceptor, and FRET) are shown. The calculated colocalization diagram and colocalized FRET index after the subtraction of spectral bleed-through. Alexander, HepG2, and Huh7 cells were treated with a 50 nM concentration of 6HB labeled with FRET reporter dyes for 24 h. Nuclei were counterstained with Hoechst 33342 (Thermo Fisher Scientific). Confocal images were taken and analyzed for FRET using the “FRET and colocalization analyzer” ImageJ plug-in. 115 “Colocalized FRET index” images present the calculated amount of FRET for each pixel in the FRET channel. The ImageJ plug-in color codes the relative FRET efficiency ranging from blue (none FRET efficiency) to red-yellow (high FRET efficiency). The “Colocalization diagram” plots display pixel colocalization as well as color coded FRET efficiency in a 2D plot.

FIG. 19A-I show colocalization analysis of different DNs. FIG. 19A-C show Huh7 cells were treated with different types of DNs (at 50 nM concentration) for 6 h either in full medium (10% FBS EMEM) or in serum-free medium (0% FBS EMEM). After incubation, cells were labeled with lysosomal marker LysoTracker Blue DND-22 (Thermo Fisher Scientific). Stained cells were imaged using spinning disk confocal microscopy IXplore SpinSR (Olympus, Tokyo, Japan). The Pearson's correlation coefficient for fluorophore pairs either (FIG. 19B) DNA-Lysosomes or (FIG. 19C) DNA-pHrodo was calculated using the Coloc 2 tool available in ImageJ software (NIH) and is presented as means of n=30 cells. (***) P<0.001 denotes significant differences. FIG. 19D-F show HepG2 cells were treated with different types of DNs (at 50 nM concentration) for 6 h either in full medium (10% FBS EMEM) or in serum-free medium (0% FBS EMEM). After incubation, cells were labeled with lysosomal marker LysoTracker Blue DND-22 (Thermo Fisher Scientific). Stained cells were imaged using spinning disk confocal microscopy IXplore SpinSR (Olympus, Tokyo, Japan). The Pearson's correlation coefficient for fluorophore pairs eithers (e) DNA-Lysosome or (f) DNA-pHrodo was calculated using the Coloc 2 tool available in ImageJ software (NIH) and is presented as means of n=30 cells. (***) P<0.001 denotes significant differences. FIG. 19G-I show Alexander cells were treated with different types of DNs (at 50 nM concentration) for 6 h either in full medium (10% FBS EMEM) or in serum-free medium (0% FBS EMEM). After incubation cells were labeled with lysosomal marker LysoTracker Blue DND-22 (Thermo Fisher Scientific). Stained cells were imaged using spinning disk confocal microscopy IXplore SpinSR (Olympus, Tokyo, Japan). The Pearson's correlation coefficient for fluorophore pairs either (FIG. 19H) DNA-Lysosomes or (FIG. 19I) DNA-pHrodo was calculated using the Coloc 2 tool available in ImageJ software (NIH) and is presented as means of n=30 cells. (***) P<0.001 denotes significant differences.

FIG. 20 shows colocalization assessment of scrambled aurein-decorated DNs in three distinct hepatic cell lines. Alexander, HepG2, Huh7 cell lines were treated with 50 nM concentration of different DNs for 6 h either in full medium (10% FBS EMEM) or in serum-free medium (0% FBS EMEM). After incubation cells were labelled with lysosomal marker LysoTracker® Blue DND-22 (Thermo Fisher Scientific). Stained cells were imaged using spinning disk confocal microscopy IXplore SpinSR (Olympus, Tokyo, Japan). Representative images out of three independent experiments are presented. DNs possess pHrodo (red) dye. DNA of DNs was labelled with Alexa-488.

FIG. 21 shows Pearson's correlation coefficient for fluorophore pairs either DNA-Lysosomes or DNA-pHrodo was calculated using Coloc 2 tool available in ImageJ software (NIH) and presented as means of n=30 cells.

FIG. 22A-C show DN-protein interaction. FIG. 22A shows different types of DNs at concentration 50 nM were incubated either in HBSS or in EMEM medium (ATCC) supplemented with 10% fetal bovine serum (FBS, Thermo Fisher Scientific) for 2 h at 37° C. The DNs were centrifuged and washed with PBS. Elution and denaturation in sample loading buffer was used to detach proteins associated with the particles. Afterward, proteins were separated by gel electrophoresis. Gels were stained with Coomassie blue (AppliChem). FIG. 22B-C show analyses of the protein corona on the particles assessed by Fluorescence Correlation Spectroscopy (FCS). Different types of DNs were incubated either in HBSS, or in EMEM medium (ATCC) supplemented with 10% fetal bovine serum (FBS, Thermo Fisher Scientific), and the mean diffusion time was measured by FCS. FIG. 22B shows a Table summarizing diffusion times of different DNs incubated in different buffer conditions in milliseconds. The data are presented as mean±SE, n=3. The mean diffusion time is given in milliseconds (ms). FIG. 22C shows examples of autocorrelation curves obtained for the diffusion of fluorescently labeled particles in EMEM medium supplemented with 10% fetal bovine serum. The measurements were performed immediately after adding the particles to the medium and after 60 min after addition.

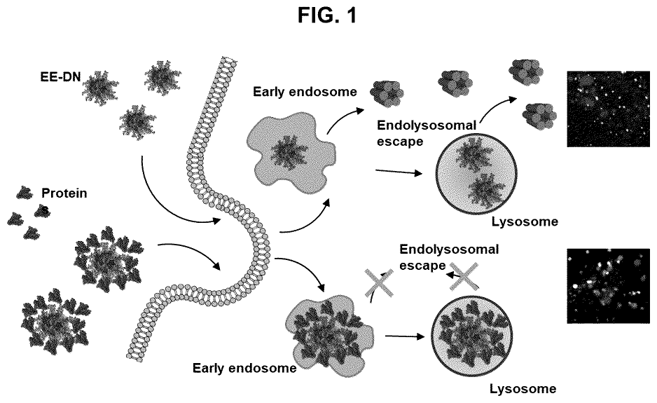

### DETAILED DESCRIPTION

Unless otherwise defined, all technical and scientific terms used herein have the same meaning as commonly understood by one of ordinary skill in the art. For example, any nomenclatures used in connection with, and techniques of, cell and tissue culture, molecular biology, immunology, microbiology, genetics, and protein and nucleic acid chemistry and hybridization described herein are well known and commonly used in the art. In case of conflict, the present disclosure, including definitions, will control. Exemplary methods and materials are described below, although methods and materials similar or equivalent to those described herein can be used in practice or testing of the embodiments and aspects described herein.

As used herein, the terms “amino acid,” “nucleotide,” “polynucleotide,” “vector,” “polypeptide,” and “protein” have their common meanings as would be understood by a biochemist of ordinary skill in the art. Standard single letter nucleotides (A, C, G, T, U) and standard single letter amino acids (A, C, D, E, F, G, H, I, K, L, M, N, P, Q, R, S, T, V, W, or Y) are used herein.

As used herein, the terms such as “include,” “including,” “contain,” “containing,” “having,” and the like mean “comprising.” The present disclosure also contemplates other embodiments “comprising,” “consisting of,” and “consisting essentially of,” the embodiments or elements presented herein, whether explicitly set forth or not.

As used herein, the term “a,” “an,” “the” and similar terms used in the context of the disclosure (especially in the context of the claims) are to be construed to cover both the singular and plural unless otherwise indicated herein or clearly contradicted by the context. In addition, “a,” “an,” or “the” means “one or more” unless otherwise specified.

As used herein, the term “or” can be conjunctive or disjunctive.

As used herein, the term “substantially” means to a great or significant extent, but not completely.

As used herein, the term “about” or “approximately” as applied to one or more values of interest, refers to a value that is similar to a stated reference value, or within an acceptable error range for the particular value as determined by one of ordinary skill in the art, which will depend in part on how the value is measured or determined, such as the limitations of the measurement system. In one aspect, the term “about” refers to any values, including both integers and fractional components that are within a variation of up to ±10% of the value modified by the term “about.” Alternatively, “about” can mean within 3 or more standard deviations, per the practice in the art. Alternatively, such as with respect to biological systems or processes, the term “about” can mean within an order of magnitude, in some embodiments within 5-fold, and in some embodiments within 2-fold, of a value. As used herein, the symbol “~” means “about” or “approximately.”

All ranges disclosed herein include both end points as discrete values as well as all integers and fractions specified within the range. For example, a range of 0.1-2.0 includes 0.1, 0.2, 0.3, 0.4 . . . 2.0. If the end points are modified by the term “about,” the range specified is expanded by a variation of up to +10% of any value within the range or within 3 or more standard deviations, including the end points.

As used herein, the terms “active ingredient” or “active pharmaceutical ingredient” refer to a pharmaceutical agent, active ingredient, compound, or substance, compositions, or mixtures thereof, that provide a pharmacological, often beneficial, effect.

As used herein, the terms “control,” or “reference” are used herein interchangeably. A “reference” or “control” level may be a predetermined value or range, which is employed as a baseline or benchmark against which to assess a measured result. “Control” also refers to control experiments or control cells.

As used herein, the term “dose” denotes any form of an active ingredient formulation or composition, including cells, that contains an amount sufficient to initiate or produce a therapeutic effect with at least one or more administrations. “Formulation” and “composition” are used interchangeably herein.

As used herein, the term “prophylaxis” refers to preventing or reducing the progression of a disorder, either to a statistically significant degree or to a degree detectable by a person of ordinary skill in the art.

As used herein, the terms “effective amount” or “therapeutically effective amount,” refers to a substantially non-toxic, but sufficient amount of an action, agent, composition, or cell(s) being administered to a subject that will prevent, treat, or ameliorate to some extent one or more of the symptoms of the disease or condition being experienced or that the subject is susceptible to contracting. The result can be the reduction or alleviation of the signs, symptoms, or causes of a disease, or any other desired alteration of a biological system. An effective amount may be based on factors individual to each subject, including, but not limited to, the subject's age, size, type or extent of disease, stage of the disease, route of administration, the type or extent of supplemental therapy used, ongoing disease process, and type of treatment desired.

As used herein, the term “subject” refers to an animal. Typically, the subject is a mammal. A subject also refers to primates (e.g., humans, male or female; infant, adolescent, or adult), non-human primates, rats, mice, rabbits, pigs, cows, sheep, goats, horses, dogs, cats, fish, birds, and the like. In one embodiment, the subject is a primate. In one embodiment, the subject is a human.

As used herein, a subject is “in need of treatment” if such subject would benefit biologically, medically, or in quality of life from such treatment. A subject in need of treatment does not necessarily present symptoms, particular in the case of preventative or prophylaxis treatments.

As used herein, the terms “inhibit,” “inhibition,” or “inhibiting” refer to the reduction or suppression of a given biological process, condition, symptom, disorder, or disease, or a significant decrease in the baseline activity of a biological activity or process.

As used herein, “treatment” or “treating” refers to prophylaxis of, preventing, suppressing, repressing, reversing, alleviating, ameliorating, or inhibiting the progress of biological process including a disorder or disease, or completely eliminating a disease. A treatment may be either performed in an acute or chronic way. The term “treatment” also refers to reducing the severity of a disease or symptoms associated with such disease prior to affliction with the disease. “Repressing” or “ameliorating” a disease, disorder, or the symptoms thereof involves administering a cell, composition, or compound described herein to a subject after clinical appearance of such disease, disorder, or its symptoms. “Prophylaxis of” or “preventing” a disease, disorder, or the symptoms thereof involves administering a cell, composition, or compound described herein to a subject prior to onset of the disease, disorder, or the symptoms thereof. “Suppressing” a disease or disorder involves administering a cell, composition, or compound described herein to a subject after induction of the disease or disorder thereof but before its clinical appearance or symptoms thereof have manifest.

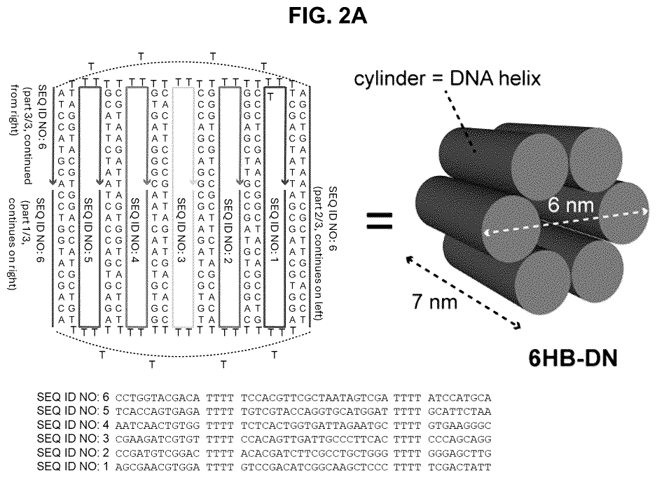

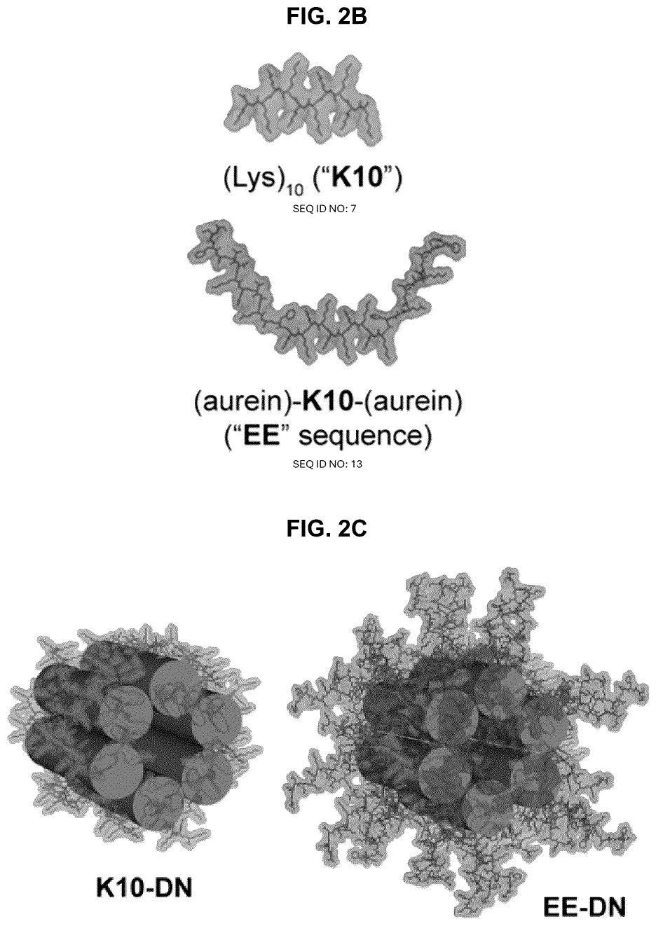

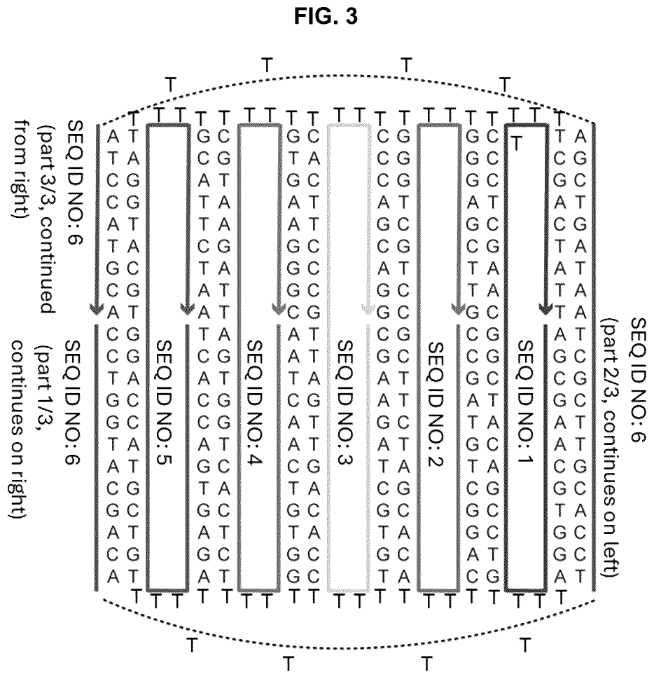

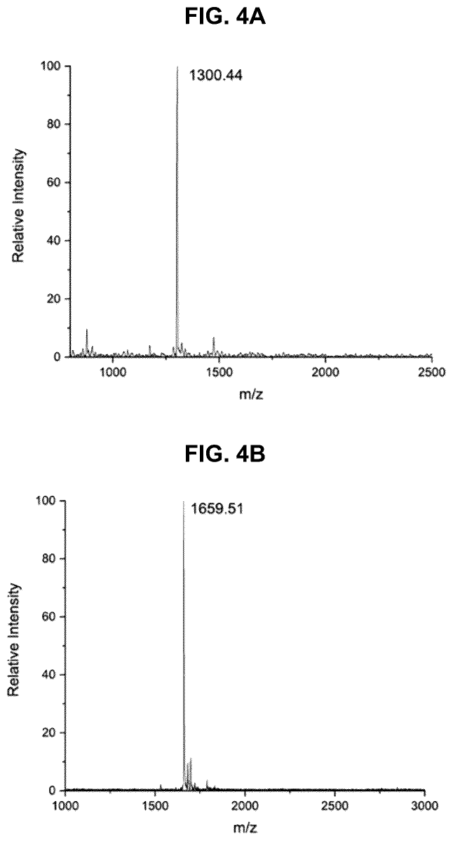

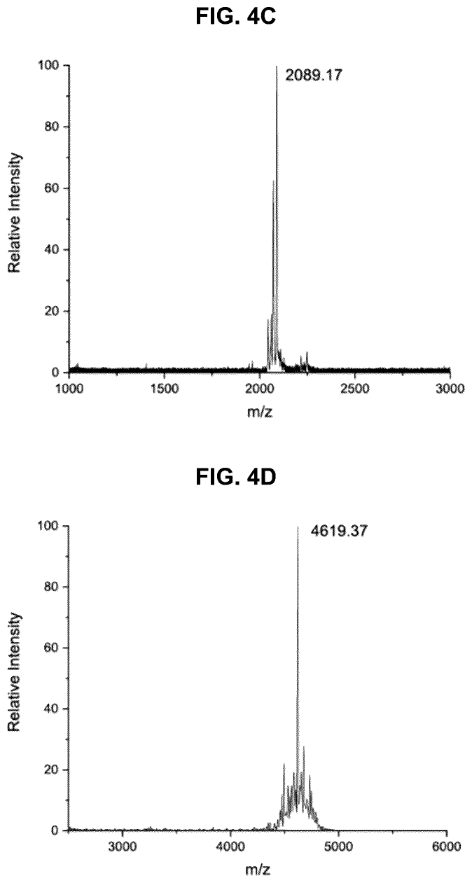

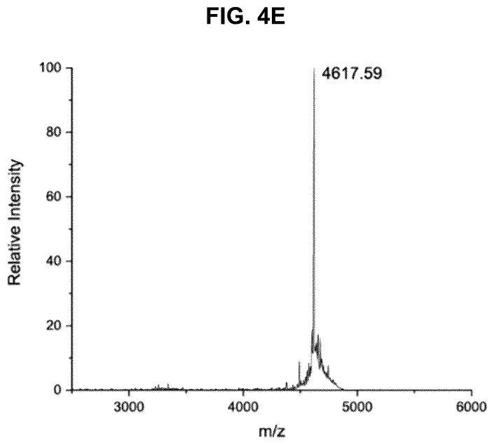

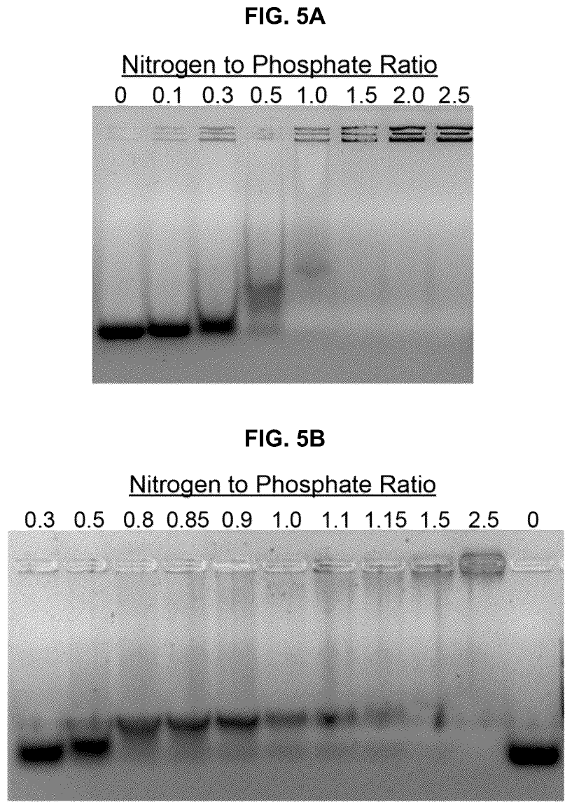

In order to probe the effect of serum proteins and DN modification on their cellular interaction, a 6-helix bundle (6HB) nanostructure was used (FIG. 2A). It was found that such bundles directly interact with cell membranes and exhibit selective interaction with distinct cell types. Additionally, the 6HB may remodel lipid membranes and mediate the formation of nanopores. However, their ability to facilitate endosomal escape in living cells is unknown, and to current knowledge, no DN has been functionalized with an endosome escape peptide to impart this activity. This structure has the advantage of simplicity (being composed of only six strands), ease of formation through a simple annealing process, and high yield. The bundle is also a rigid and monomeric assembly roughly 7×6 nm² in size. In addition to the bare 6HB nanostructures, the effect of cationic oligolysine peptide coatings was also explored, as initially reported by Shih and co-workers, to both stabilize the nanostructures to biological media conditions and to functionalize them with bioactive peptides. Toward this end, a (Lys)₁₀ peptide (K10, SEQ ID NO: 7) was synthesized, as well as a peptide flanking K10 with two copies of a sequence termed aurein 1.2 (SEQ ID NO: 12), which was found to facilitate endosomal escape (vide infra). The K10 and K10-[aurein 1.2]₂ (SEQ ID NO: 13 which was termed “EE”) peptides were obtained by solid-phase peptide synthesis (see FIG. 3-4, and Tables 1 and 2) and used to coat the 6HB nanostructures (FIG. 2B-C). As evidenced by native agarose gel electrophoresis, the peptides were able to most effectively coat the nanostructures though electrostatic interactions at a nitrogen/phosphate (N/P) ratio of ~1 (FIG. 5). It is worth noting that some aggregation occurs in both K10 and EE structures (FIG. 5).

TABLE 1
Sequences of the DNA strands that make up the 6-helix bundle DN.
The colors correspond to the strands in FIG. 3.

| Name | DNA Sequence (5′→3′) | SEQ ID NO |
|---|---|---|
| 6HB-Blue | AGCGAACGTGGATTTTGTCCGACATCGGCAAGCTCCCTTTTTCGAC | 1 |
| | TATT | |
| 6HB-Green | CCGATGTCGGACTTTTACACGATCTTCGCCTGCTGGGTTTTGGGAG | 2 |
| | CTTG | |
| 6HB-Yellow | CGAAGATCGTGTTTTTCCACAGTTGATTGCCCTTCACTTTTCCCAG | 3 |
| | CAGG | |
| 6HB-Orange | AATCAACTGTGGTTTTTCTCACTGGTGATTAGAATGCTTTTGTGAA | 4 |
| | GGGC | |
| 6HB-Red | TCACCAGTGAGATTTTTGTCGTACCAGGTGCATGGATTTTTGCATT | 5 |
| | CTAA | |
| 6HB-Purple | CCTGGTACGACATTTTTCCACGTTCGCTAATAGTCGATTTTATCCA | 6 |
| | TGCA-Alexa Fluor 488 | |

TABLE 2
Sequences of the synthesized peptides along with their expected masses. The
observed mass was obtained using MALDI-MS, the spectra are displayed
in FIG. 4.

| | | Expected | SEQ |
|---|---|---|---|
| Peptide | Amino Acid Sequence | Mass | ID NO |
| K10 | KKKKKKKKKK | 1298.75 | 7 |
| K10 with fluorescein | Fluorescein-KKKKKKKKKK | 1657.05 | 8 |
| K10 with pHrodo Red | KKKKKKKKKKC-pHrodo Red | ≈2108.9* | 9 |
| EE-K10 | GLFDIIKKIAESFGSGKKKKKKKKKKGSGFE | 4626.65 | 10 |
| | AIKKIIDFLG | | |
| EE scramble-K10 | IKAFKGFDESILIGSGKKKKKKKKKKGSGIL | 4626.65 | 11 |
| | ISEDFGKFAKI | | |
| aurein 1.2 | GLFDIIKKIAESF | — | 12 |
| K10-[aurein 1.2]₂ | GLFDIIKKIAESFKKKKKKKKKKGLFDIIKKIAE | — | 13 |
| (EE) | SF | | |

*the pHrodo dye from the supplier was reported as a mass of “~700” Da, so this value was used to calculate the expected mass of the peptide-dye conjugate,

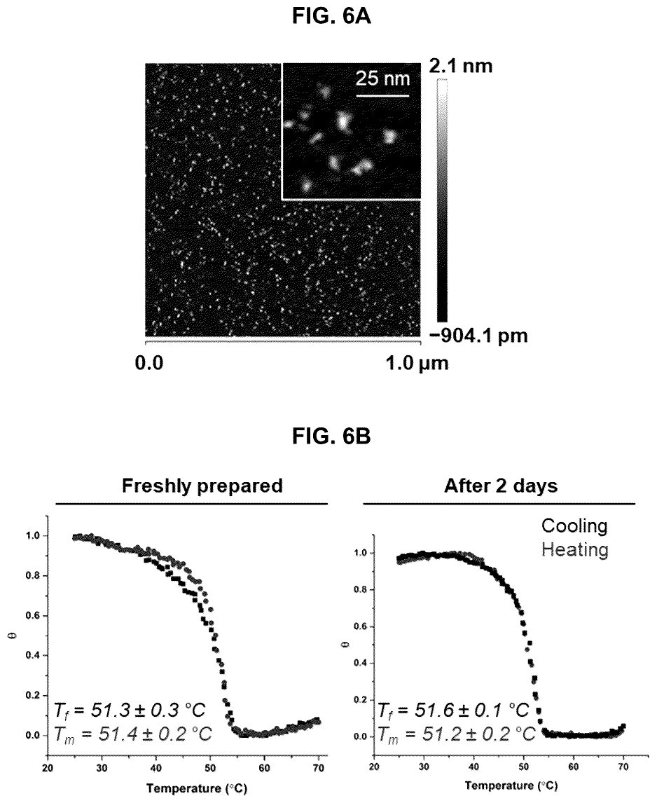

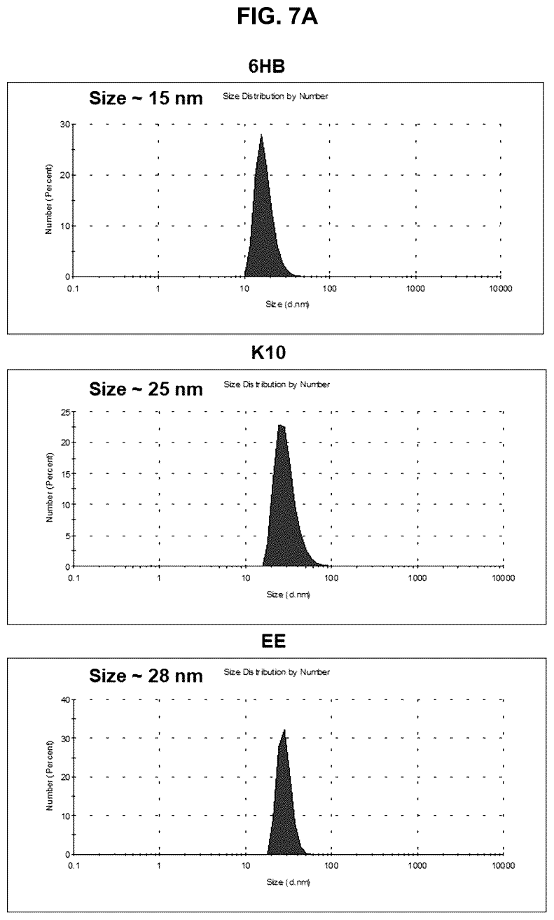

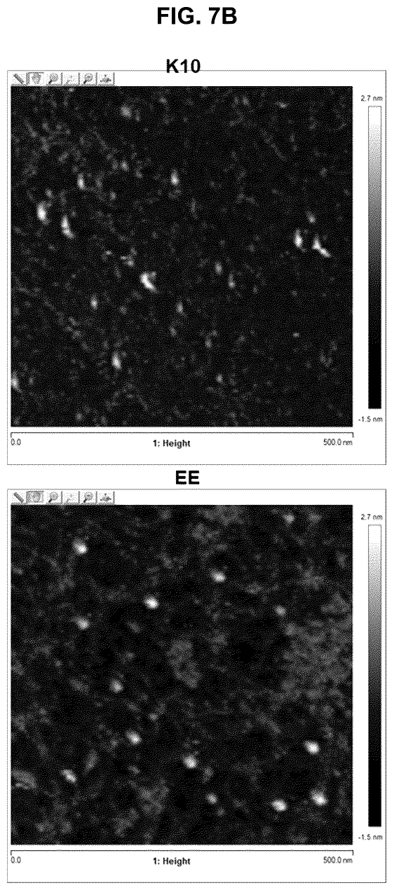

However, subsequent atomic force microscopy (AFM) and dynamic laser light scattering (DLS) analysis revealed that this aggregation is very minor (FIG. 6A and FIG. 7). AFM imaging was used to visualize the 6HB structures (FIG. 6A) and saw that samples were primarily monodispersed, with a minimal amount of aggregates. DLS analysis in aqueous solution revealed distinct mean hydrodynamic diameters of about 15, 25, and 28 nm for 6HB, K10, and EE (FIG. 7), respectively, where it is surmised that the greater diameter for the latter two structures corresponds to the peptide coatings. These data confirmed the theoretically estimated DN sizes (FIG. 2).

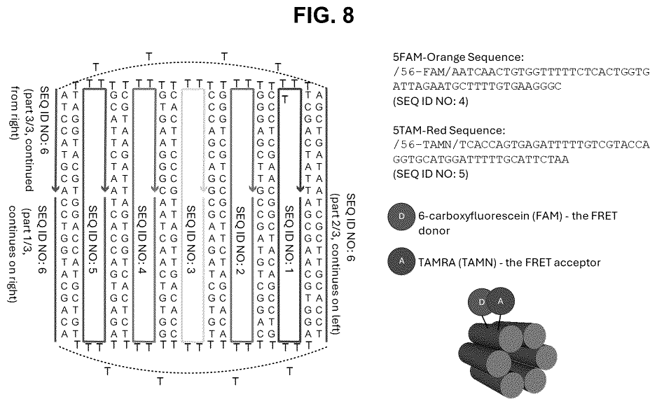

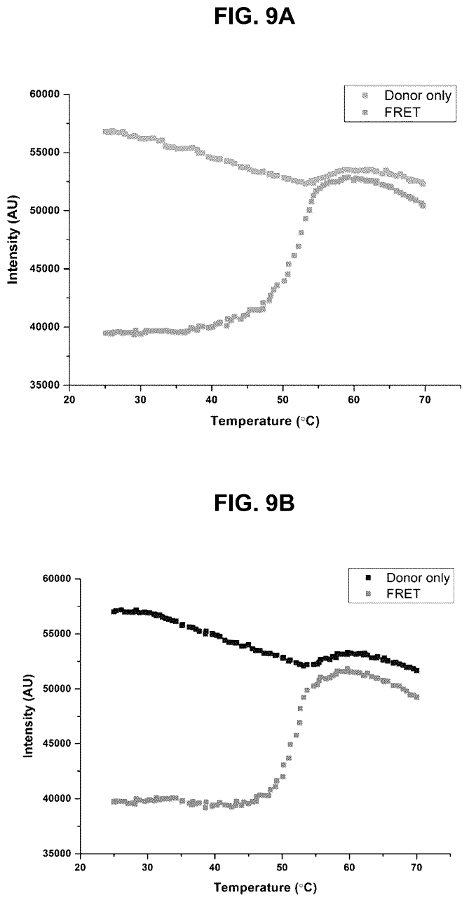

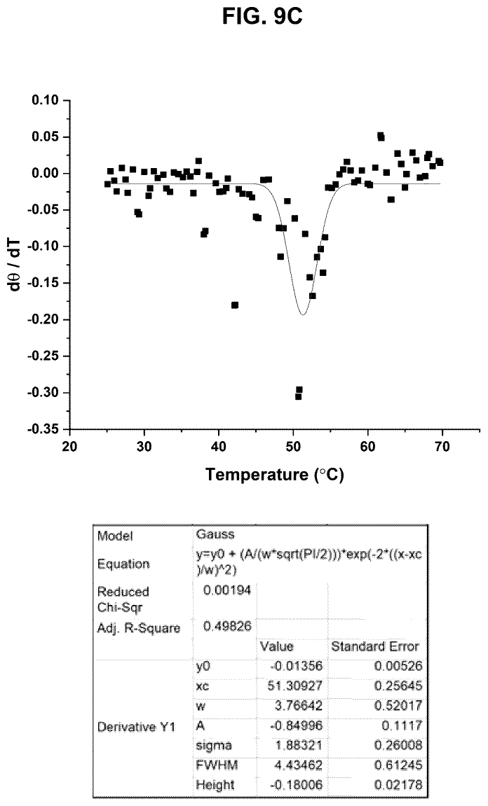

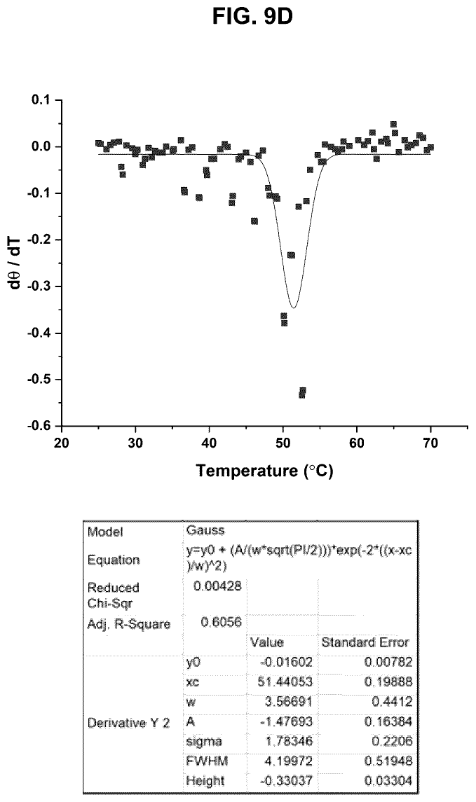

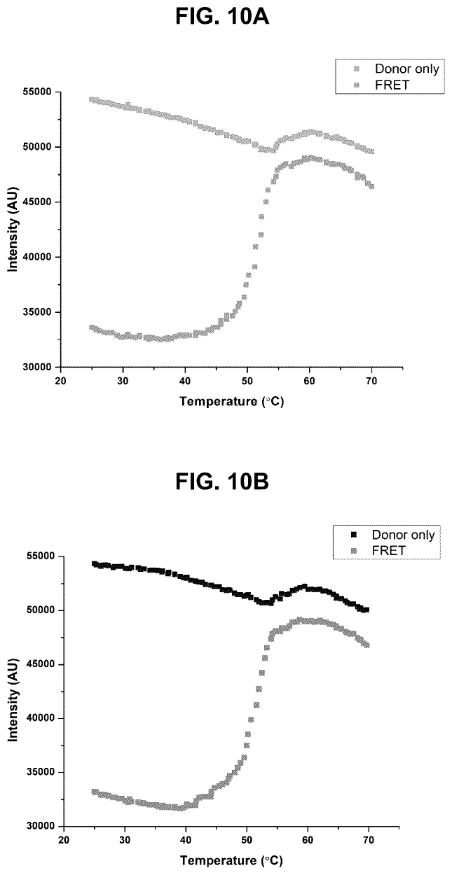

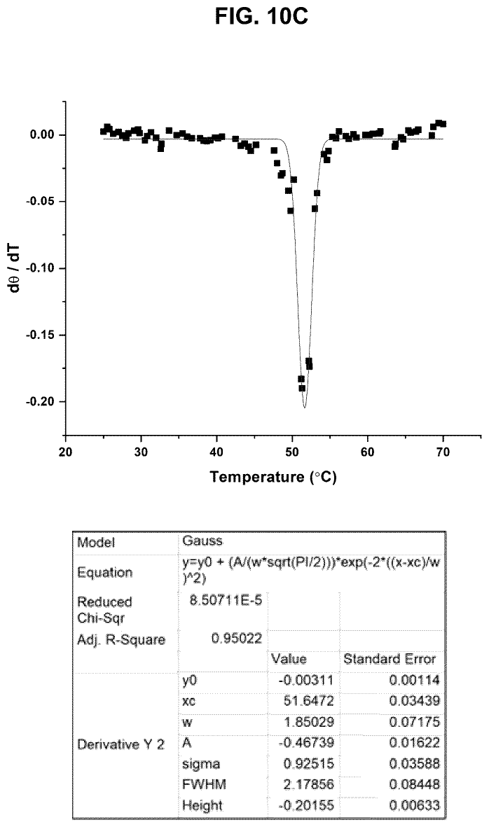

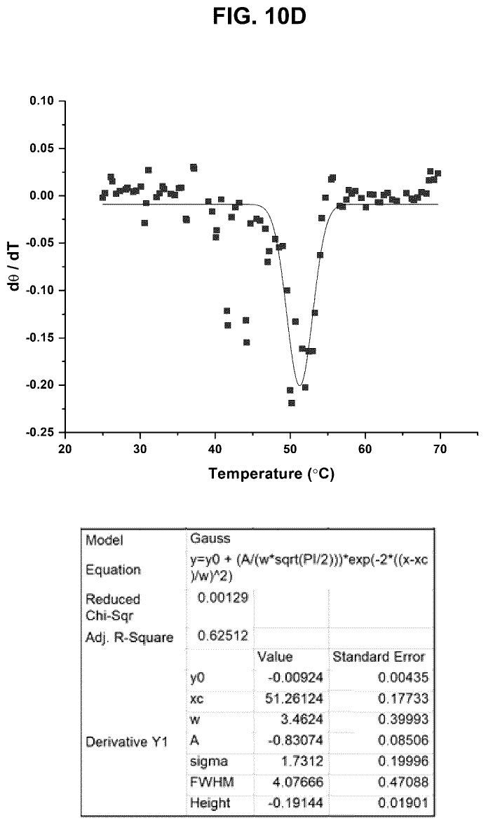

Multiple studies have shown that various DNA nanostructures remain substantially intact in different physiological media and even within cells for at least 24 h. However, the stability of DNs greatly depends on multiple parameters, e.g., temperature, exposure time, and DN design. Therefore, the structural stability of DNA nanostructures in physiological buffer (PBS) was assessed. A previously reported temperature-induced unfolding assay for DNs was utilized. This assay relies on measuring of the transition temperatures (the temperature of folding (T_{f}) and the temperature of melting (Tₘ) by monitoring fluorescence resonance energy transfer (FRET) between two incorporated dyes upon heating. The analysis of T_{f} and Tₘ serves as a tool to reveal the local structural changes of DNA nanostructures in detail. When DNA nanostructures are intact the FRET pairs are held in close contact, leading to a high FRET efficiency. Conversely, the melting and disassembly of DNA nanostructures result in increased donor-acceptor distances and a subsequent decrease of FRET efficiency. 6HB structures (FIG. 8) containing the FRET reporter dyes (donor, 6-carboxyfluorescein; acceptor, TAMRA) were designed. The melting analysis revealed that the transition temperature (T_{f} and Tₘ) values were approximately equal in freshly prepared 6HB structures and incubated in PBS for 2 days (FIG. 6B and FIG. 10-11) and were approximately 51° C. This data suggest that the DNA nanostructures remained stable and assembled under physiological conditions and buffers.

Accumulating evidence suggests that, upon intravenous injection, the majority of nanomaterials are ultimately sequestered by the liver. Additionally, nanomaterials have been shown to directly interact with hepatocytes and not only Kupffer cells (liver resident macrophages). Hence, it is crucial to study the DN properties that might accelerate or obstruct their uptake by hepatocytes. Surprisingly, there is no data on DN-hepatocyte interactions in the current literature. Hepatic cell lines of varying degrees of differentiation have frequently been used to model hepatocyte functions, since primary tissue hepatocytes cannot be readily expanded ex vivo. Thus, in this study, DN-cell interactions were assessed utilizing three commonly used hepatic cell lines: HepG2, Huh7, and Alexander cells.

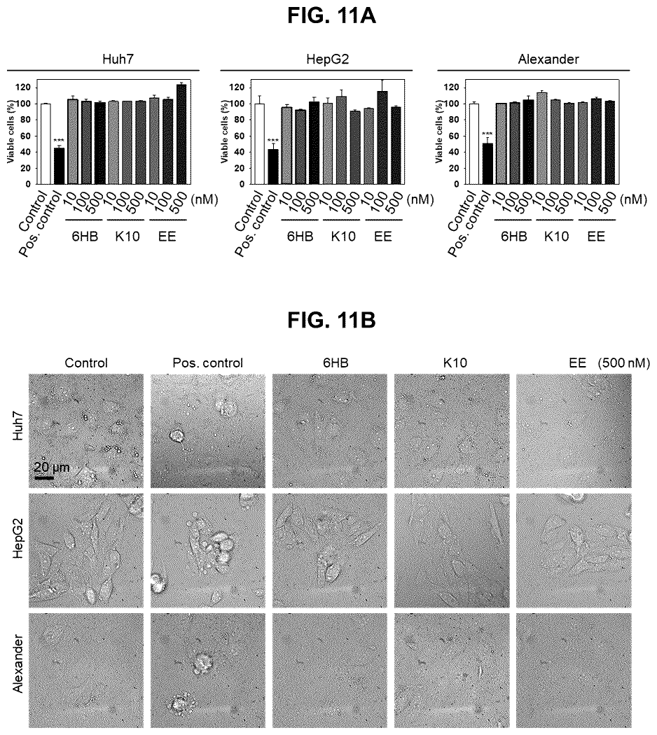

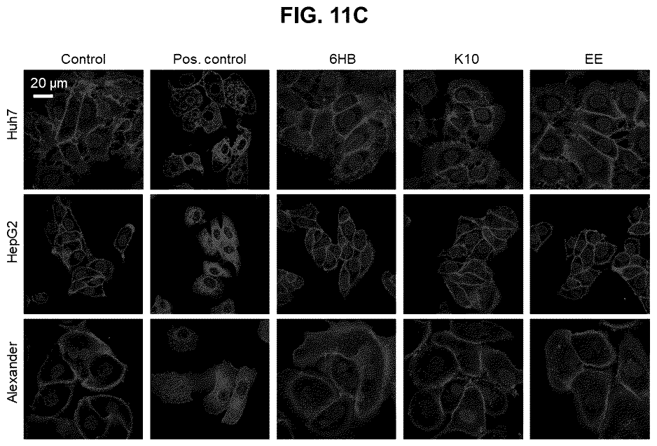

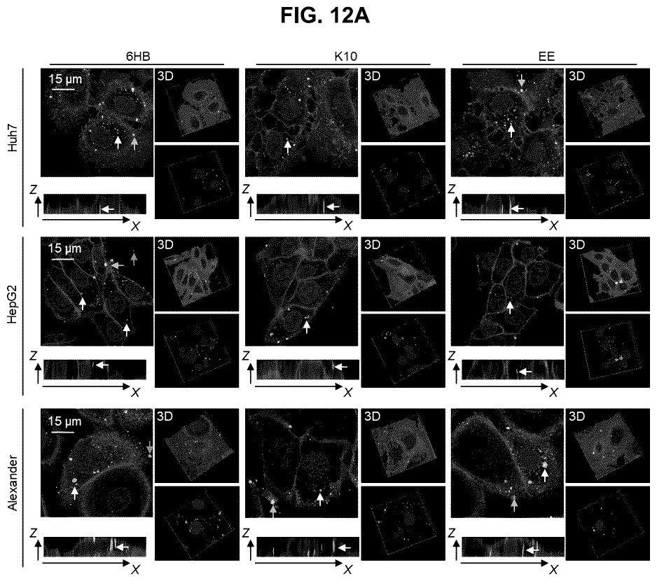

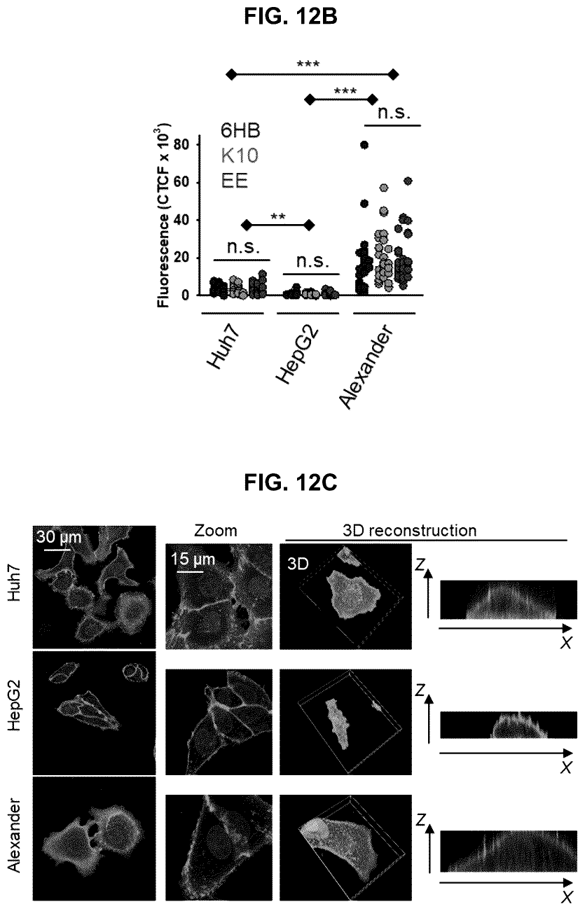

To ensure that DNs do not induce any toxic effects on the cells during the experiments, the effects of different DN types on cell viability were first analyzed. Huh7 or HepG2 as well as Alexander cells cultured in medium for 24 h in the presence or absence of 6HB, or the K10- and EE-coated nanostructures, showed no decrease in cell viability (FIG. 11A). Additionally, the treatment of all three cell lines with different DN types did not result into any noticeable morphological changes (FIG. 11B-C). On the contrary, treatment with ethanol, used as a positive control, led to marked membrane rupture as evident by dye cytosolic accumulation and massive vesicle shedding (FIG. 11C). Further, the uptake of differently functionalized DNs by three cell lines were analyzed. To enable tracking of cellular uptake, one strand comprising the DNs was labeled with AlexaFluor-488. First, an end-point uptake study was conducted to examine and compare different DN cellular uptakes in all cell lines, which was characterized qualitatively and quantitatively by high-resolution spinning disc confocal microscopy. Incubation for 24 h with 50 nM of different types of DNs led to a noticeable intracellular accumulation of the nanostructures in all three cell types (FIG. 12A). DNs exhibited different internalization behaviors in different cell lines (FIG. 12B). Alexander cells showed a significantly higher cellular uptake efficiency of DNs compared to Huh7 and HepG2 (FIG. 12B). HepG2 were the least effective in engulfing DNs (FIG. 12B). However, no significant differences in the uptake of differently functionalized DNs within the same cell line were found (FIG. 12B). All uptake data is presented as corrected total cell fluorescence (CTCF) 74 of the full area of interest to average a single cell fluorescence measured by confocal microscopy. CTCF represents the sum of pixel intensity for a single image with the subtraction of average signal per pixel for a background region. The detailed description of CTCF calculations is presented in the Experimental Section. To ensure that the cell membrane-bound DNs were removed from quantification and only internalized DNs were considered in calculations, cell membrane counterstaining was performed and the 3D microscopic image analysis of the particle internalization (FIG. 12A). From the orthogonal sections in the x-z planes, one can estimate that measurements in the x-y plane, performed at a z-position of about half height of the cell, enable reliable discrimination of the membrane-associated or intracellular DNs (FIG. 12A, white and grey arrows).

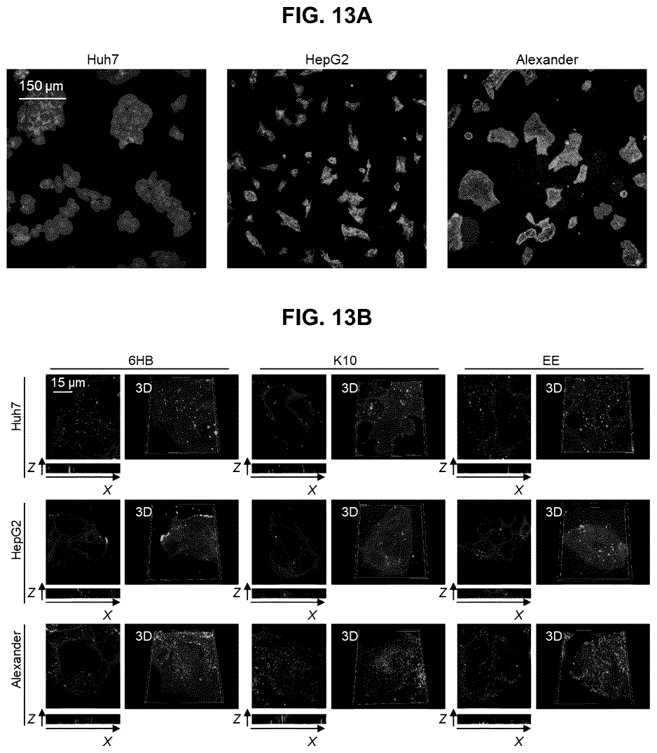

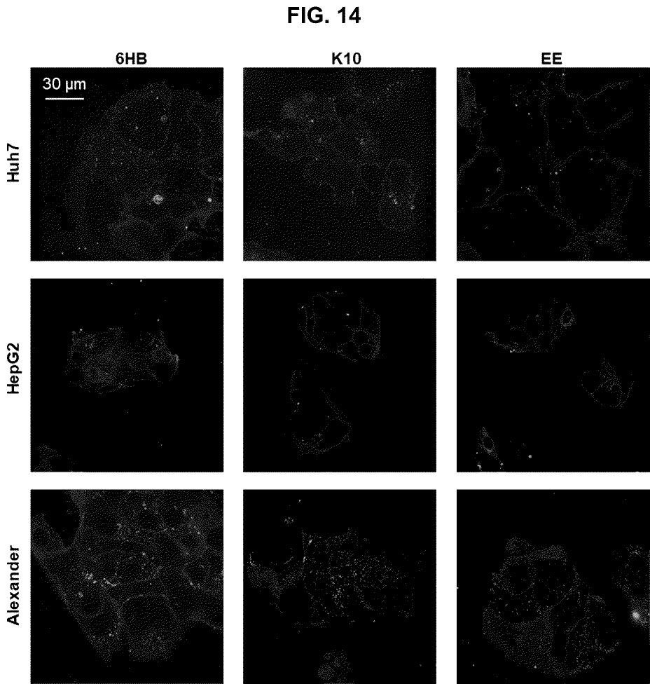

In fact, it is known that even closely related cell lines respond differently to external stimuli. Specifically, NP uptake may dramatically differ in distinct cell lines of the same lineage. Additionally, cell geometry and morphology have been recognized as important factors affecting cell behavior and intracellular trafficking. Given these factors, the cellular morphology of HepG2, Huh7, and Alexander cells was further analyzed. Confocal microscopy revealed that cells of different lines are distinct in size and morphology (FIG. 12C and FIG. 12A). HepG2 showed an elongated shape, whereas Huh7 bore a cuboidal epithelial-like morphology (FIG. 12C). Alexander cells had a hexagonal epithelial-like morphology (FIG. 12C). Further, using a distinct membrane labeling dye, it was confirmed that 24 h treatment with 50 nM of different types of DNs resulted in sufficient intracellular accumulation of the nanostructures in all three cell types (FIGS. 13B and 14).

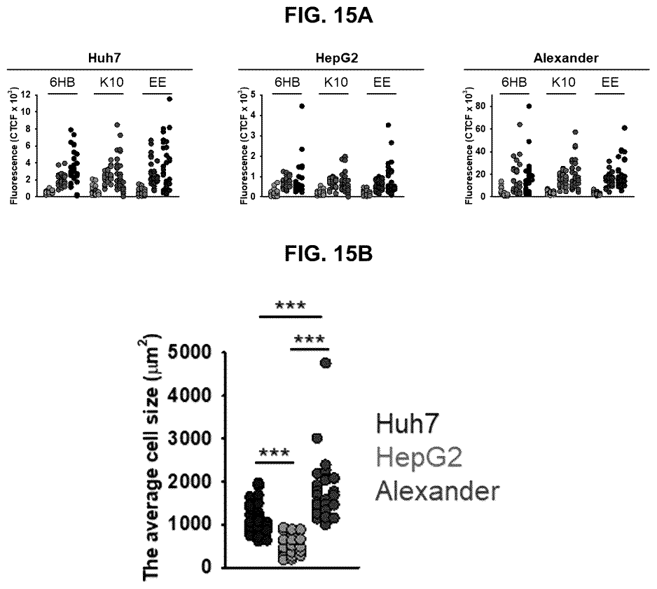

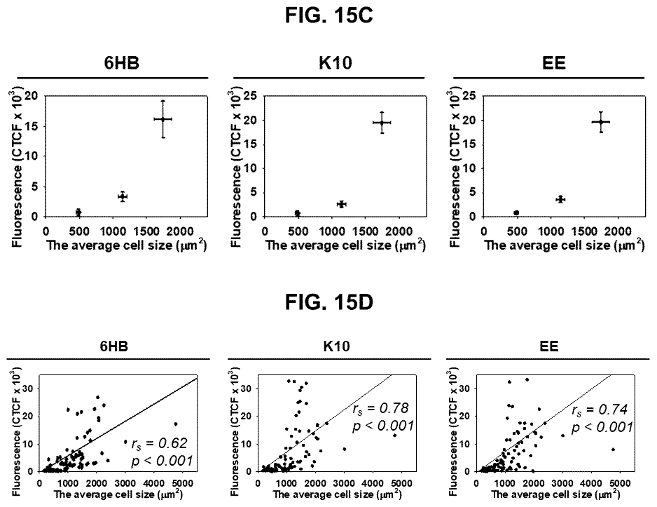

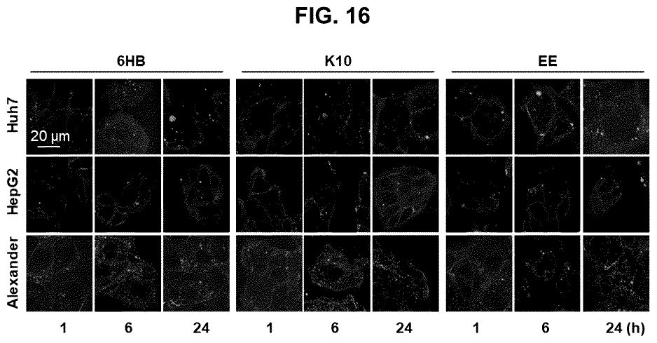

Next, a time-course study (1, 6, and 24 h) was conducted to examine and compare DN cellular uptake over time. The analysis of the uptake of three types of DNs by HepG2, Huh7, and Alexander cells by quantitative confocal microscopy revealed that within 1 h all types of DNs were engulfed by all three cell lines (FIG. 15A and FIG. 16). However, the uptake process did not stop at the 1 h time point and continued up to 24 h of incubation (FIG. 15A and FIG. 16). Consistent with end-point analysis (FIG. 12A), a time-course study (FIG. 15A) revealed that Alexander cells showed the highest extent of DN internalization. It was shown that all three cell lines are distinct in size and morphology (FIG. 12C). Further, the size differences between cell lines was quantitatively assessed, revealing that the average area of Alexander cells is about 1700 μm², that of Huh7 is 1100 μm², and the of HepG2 is 500 μm² (FIG. 15B). It becomes evident that cell size plays a central role to many cellular functions. Of note, cell size has been identified as a factor that determines the rate of cellular uptake of proteins, endocytic structures, and nanomaterials.

Indeed, a number of studies have been conducted to reveal endocytic recognition and engulfment of different DNs by distinct cell types. In these reports, the primary focus of the research is how either the physiochemical parameters (e.g., mass, shape, size, surface functionalization) of DNs or cell phenotype modulate the average uptake of the nanostructures. However, little attention has been paid to how the cell size or other cellular characteristics at the single-cell level might affect DN ingestion. Of note, the size of a cell plays a crucial role in determining the rate of cellular uptake of materials. Although the correlation between cell size and uptake appears to be intuitive, it is still not established exactly how the physical parameters of a single cell govern its ability to uptake particles. Additionally, for DNA nanostructures, there is still only limited literature that analyzes the correlation of cell size with DNs uptake efficacy. The average cell sizes were mapped to the corresponding fluorescence of the different types of internalized DNs (FIG. 15C). This analysis revealed a linear increase in the uptake of all three types of DNs with cell size (FIG. 15C). Spearman rank order correlation analysis confirmed that the cellular uptake of DNs follows a linear relationship with the cell size (FIG. 15D).

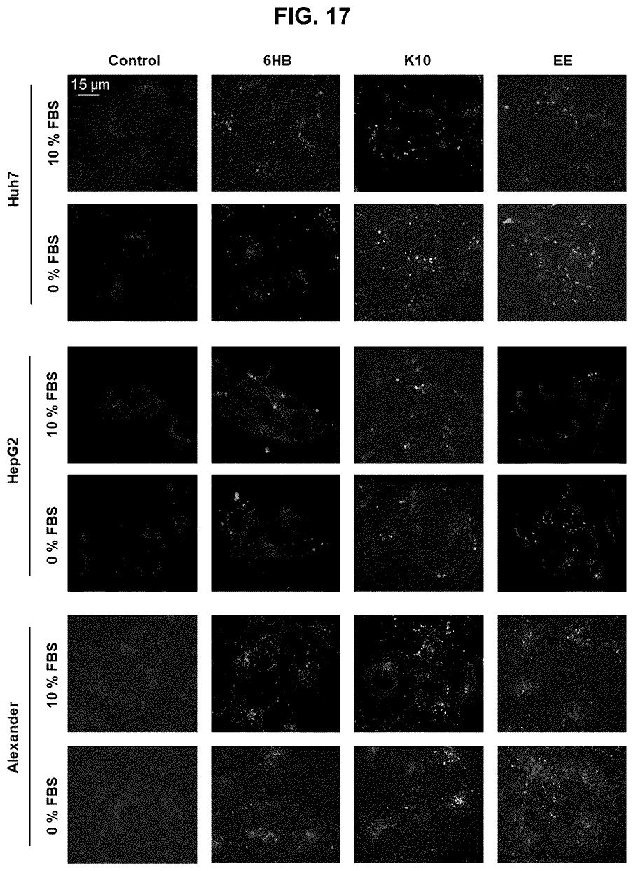

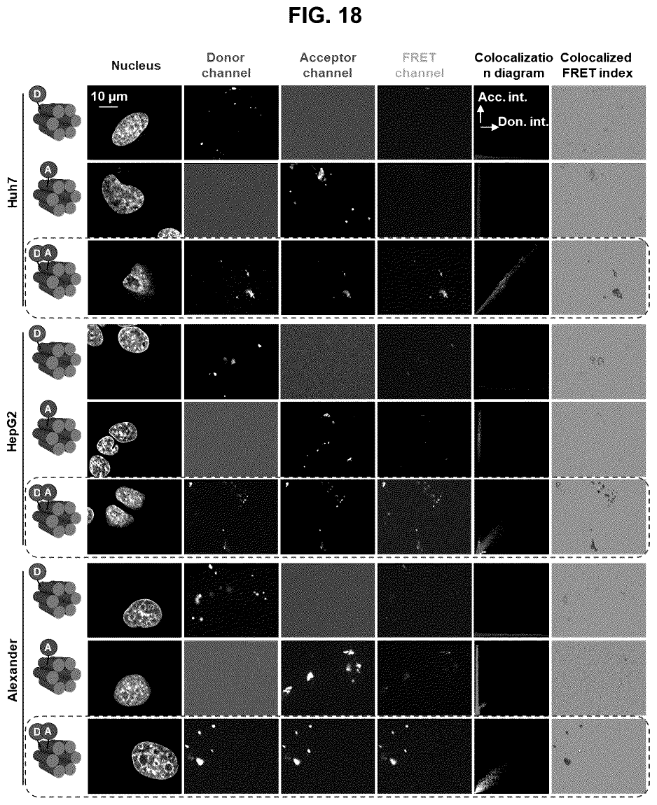

To elaborate on the key question of how the presence of serum proteins affects the desired endosome escape of DNs, it was important to first cross-check whether DNs stay intact in harsh lysosomal conditions. Indeed, the intracellular fate of DNA nanostructures remains elusive and controversial. Some evidence suggests that DNs end up in the lysosomes, whereas other studies claim that DNs accumulate in the cytosolic compartments.86 In fact, all three types of DNs were localized in the lysosomal compartments as revealed by confocal imaging (FIG. 17). Further, the stability of DNs was analyzed by utilizing FRET analysis. The above-mentioned 6HB structures containing the FRET reporter dyes (FIG. 8) were used. 6HB structures, labeled with either donor-only or acceptor-only fluorophores, served as negative controls. The Förster distance of the FRET reporter dyes used (6-carboxyfluorescein donor, TAMRA acceptor) is ~5 nm, which allows for the sensitive detection of the changes in FRET efficiency that occur during the structural changes (e.g., assembly/disassembly) of DNs. The validation of FRET by fluorescence microscopy revealed that all three cell lines, treated with a 50 nM concentration of 6HB for 24 h, showed high FRET efficiency compared with negative controls (FIG. 18). These data indicate that DNs remained largely stable in lysosomal compartments for up to 24 h of incubation.

It is worth noting here that DNs are recognized as novel smart delivery platforms for different macromolecules and drugs. Generally, the delivery of different biological agents utilizing nanobased vehicles relies on the endocytic pathway as the predominant uptake mechanism. This process leads to the entrapment of cargo inside the endosome and lysosome, where the contents can be degraded by lysosomal enzymes. In order to bypass this problem, a number of molecules and other pharmacological agents, which facilitate escape the endolysosomal compartment, have been identified. Interestingly, studies that experimentally verify endolysosomal escape are usually conducted utilizing serum-free medium. Indeed, as is well-known from nanoparticle-cell interactions, the presence of proteins and their adsorption onto a NP surface dramatically affects the resultant biological effects. Specifically, it has been shown that the protein corona substantially impairs the endolysosomal escape efficiency of different nanomaterials. Thus, it was investigated whether the protein corona has any effect on the escape efficiency of the DN functionalized with an endolysosomal escape enhancer. Bearing in mind that the uptake process continues up to 24 h (FIG. 15A and FIG. 16), an appropriate time point for endolysosomal escape assessment needed to be selected. It is well-established that serum-free cell culturing results in autophagy that dramatically biases endolysosomal interactions. However, short-term starvation up to 6 h of hepatic cells has incremental effect on autophagy, whereas 8 h and longer leads to substantial autophagic flux activation. Therefore, 6 h represented an optimal time point to monitor endolysosomal escape without concomitant autophagic flux.

Recently a 13-residue peptide, termed aurein 1.2 (GLFDIIKKIAESE) (SEQ ID NO: 12), was discovered that enhances endolysosomal escape and improved the cytosolic delivery of proteins it was appended to by up to ~5-fold. In fact, this peptide can disrupt endolysosomal membranes and in such a way trigger the escape of cargo to cytosol. Importantly, aurein facilitates endolysosomal escape without concomitant disruption of the cell membrane and does not exhibit cytotoxicity. Therefore, this peptide was used to electrostatically coat DNs (FIG. 2C) for potential enhancement of endolysosomal escape.

One of the straightforward methods to evaluate endolysosomal escape is to use microscopic imaging of fluorescently labeled materials with localization to endolysosomal compartments. Confocal fluorescence microscopy is indispensable in assessing the colocalization of labeled macromolecular species of interest. However, such analysis might be substantially hampered by undesirable phototoxic effects from laser irradiation, especially during live-cell imaging. In order to observe undamaged living cells with engulfed DNs and avoid phototoxic effects from imaging, a novel ultrafast imaging system was utilized based on the IXplore SpinSR Olympus spinning disk confocal microscope. In order to track the peptide coating and the DN separately, the DN was coated with an 80:20 ratio of the EE peptide and K10 labeled with the pH-sensitive dye pHrodo Red, which dramatically increases its fluorescence in acidic pH. It is estimated that, given the N/P ratio of these coatings and the number of phosphates in the DNA nanostructure, there are ~48 copies of the aurein 1.2 peptide per DN.

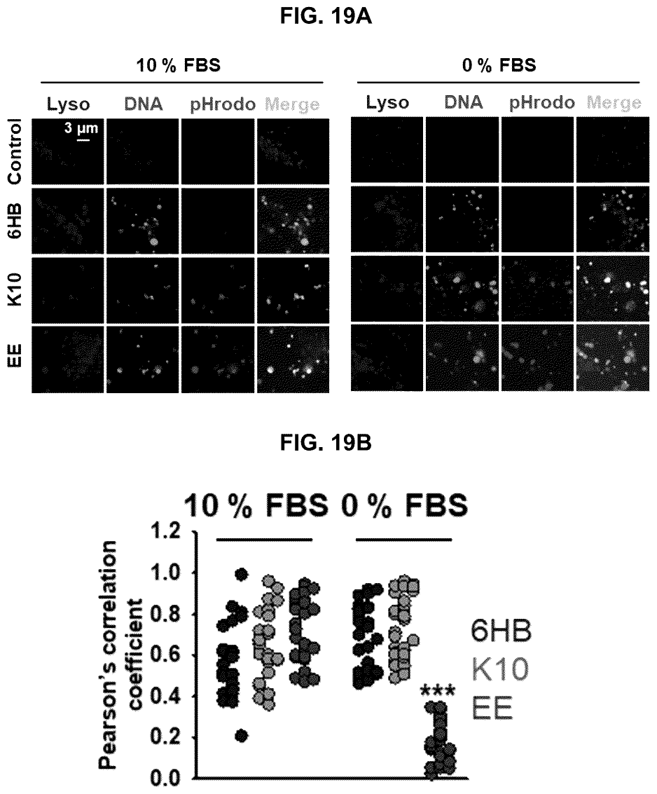

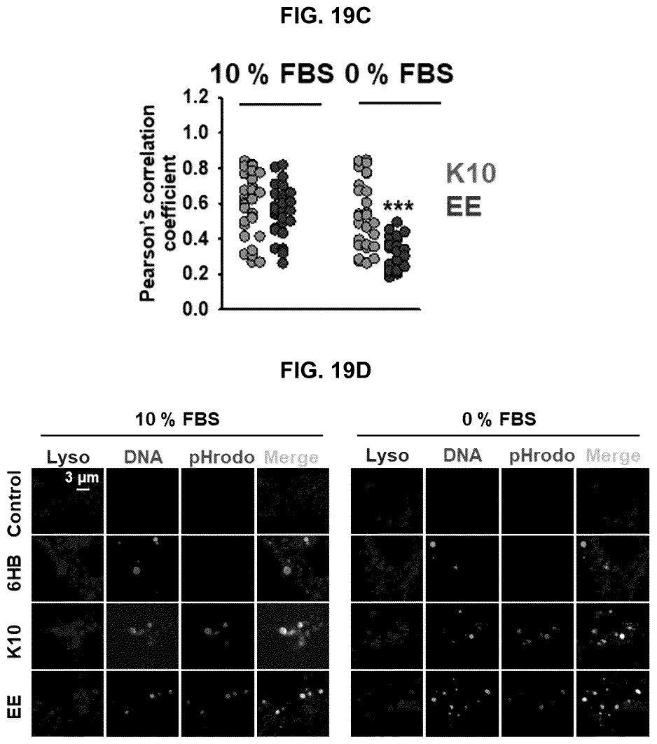

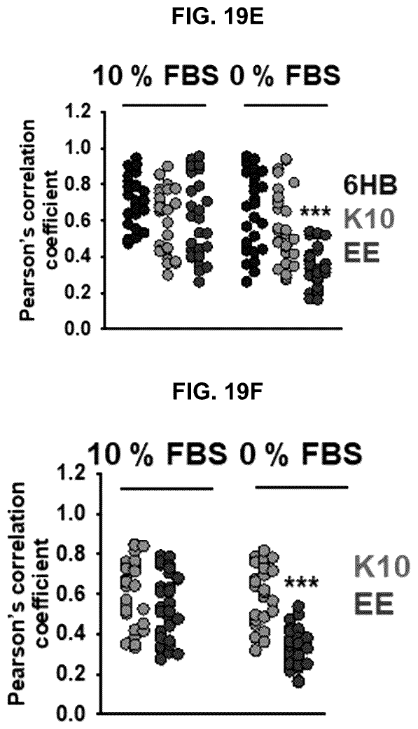

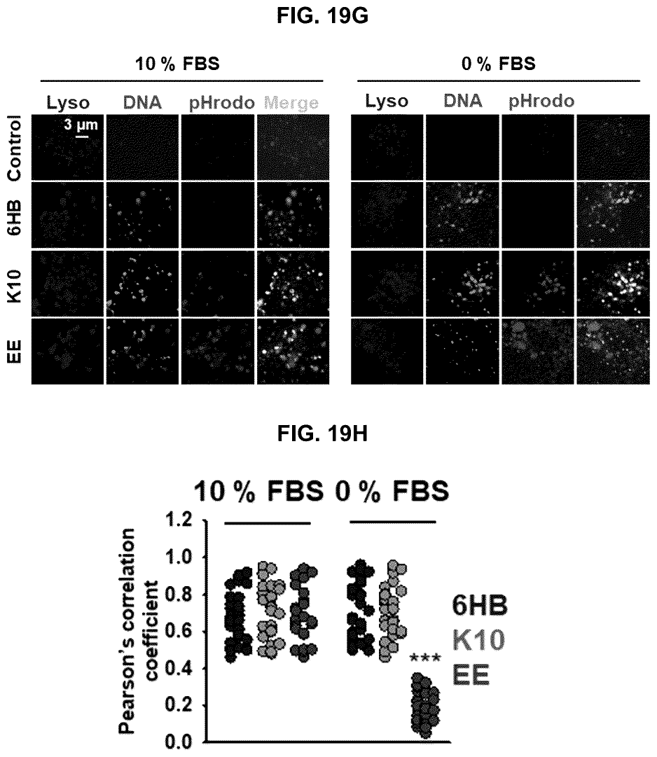

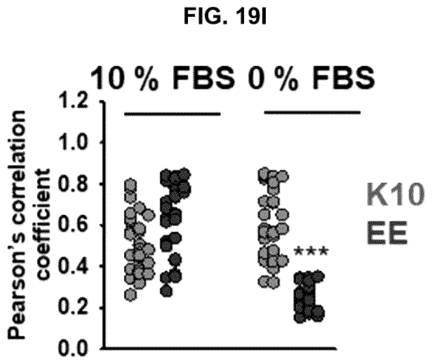

DNs with green fluorescence-labeled DNA and LysoTracker Blue DND-22 staining were used to monitor the nanostructures and endo-/lysosomes, respectively, by confocal microscopy. Indeed, aurein 1.2-decorated DNs in serum-free medium were able to escape from the endosomes/lysosomes and be released into the cytoplasm in all three cell lines after 6 h of treatment (FIG. 19 and FIG. 16). Specifically, a large amount of plain/uncoated DNs (the 6HB) accumulated in endo-/lysosomes at 6 h, while the colocalization of EE-coated DNs with endo-/lysosomes was markedly lower in all three cell lines (FIG. 19A, D, G). Additionally, the Pearson's correlation coefficient of EE-coated DN colocalization with endo-/lysosomes was below 0.5 in all three cell lines (FIG. 19B, E, H). These results suggested that a great portion of EE-coated DNs efficiently escaped from endo-/lysosomes. Further, the Pearson's correlation coefficient of DNA colocalization with pHrodo was below 0.5 for the EE-DNs in all three cell lines (FIG. 19C, F, I), suggesting that the peptide coating was probably removed from the DNA construct after endolysosomal escape.

By contrast, in the presence of serum the endolysosomal escape of EE-DNs was diminished in all three cell lines (FIG. 19A, D, G). In the presence of serum, the Pearson's correlation coefficient of EE-DN colocalization with endo-/lysosomes was substantially higher than 0.5 in all three cell lines (FIG. 19B, E, H). Moreover, there was no statistically significant difference in colocalization with endo-/lysosomes between the plain 6HB-DNs and EE-DNs (FIG. 19B, E, H). These results suggest that, in the presence of serum, the EE-DNs stayed in endo-/lysosomal vesicles during the test period. Interestingly, the Pearson's correlation coefficient of EE-DN DNA colocalization with pHrodo was higher than 0.5 in all three cell lines (FIG. 19C, F, I). These results imply that the DNA construct and aurein 1.2 peptide in the EE-DN sample did not dissociate when DNs were added to serum-containing medium.

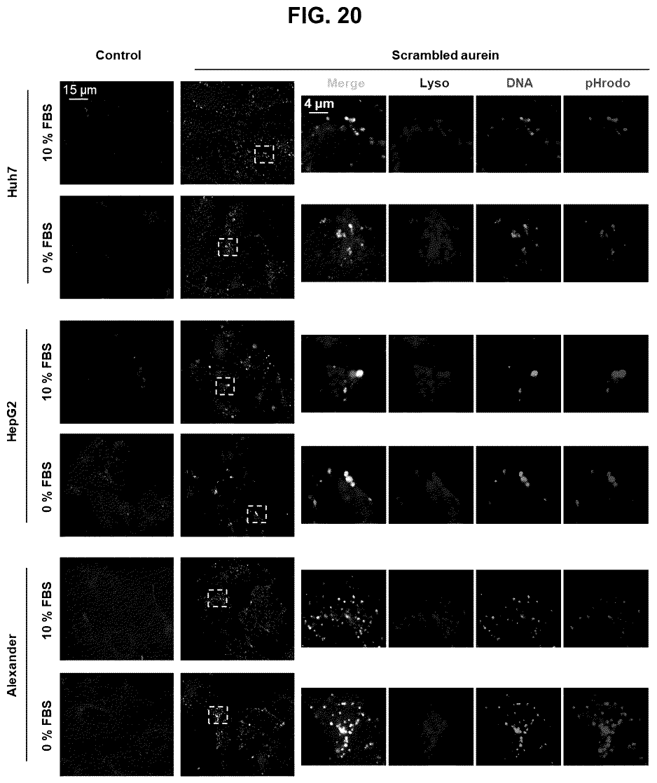

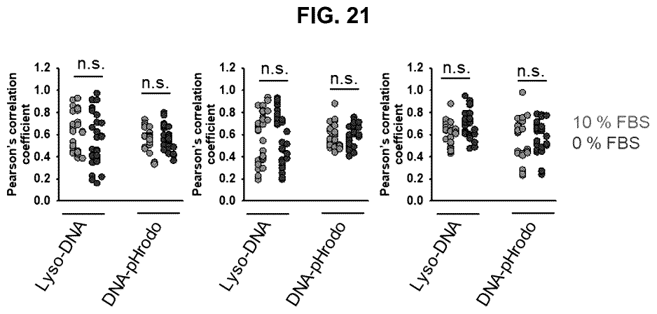

Aurein 1.2 is a derivative of so-called antimicrobial peptides, which penetrate membranes utilizing electrostatic interactions followed by the displacement of lipids and alteration of membrane structure. It was postulated that in this way antimicrobial peptides may enhance endolysosomal escape. To verify the specificity of aurein 1.2 as an endolysosomal escape enhancer, DNs were decorated with the short, highly charged peptide deca-lysine (K10). This peptide was also labeled with pHrodo Red. In fact, in neither serum-free nor serum-containing medium were K10-decorated DNs able to induce any noticeable endolysosomal escape (FIG. 19). Additionally, DNA bundles decorated with scrambled aurein 1.2 sequence were created, which were composed of the same amino acids but in a random order not expected to facilitate endolysosomal escape. In fact, those DNA bundles were unable to induce any noticeable endolysosomal escape either in the presence or absence of serum (FIG. 20-21). The data support previous findings that the effectiveness of aurein 1.2 is highly dependent on its sequence, and even closely related peptides cannot enhance endolysosomal escape to a similar extent.

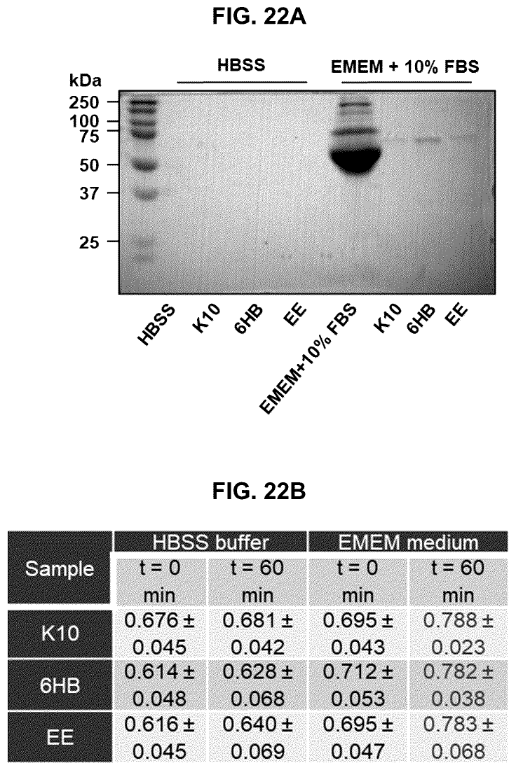

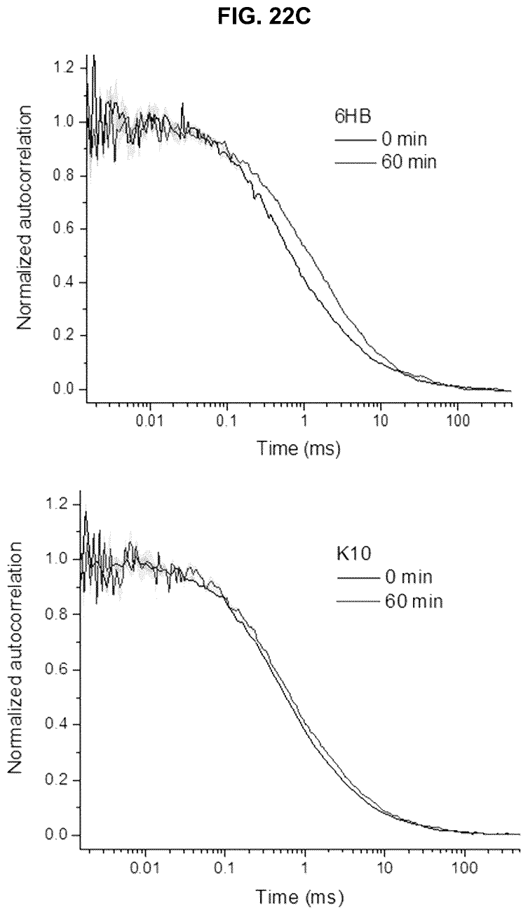

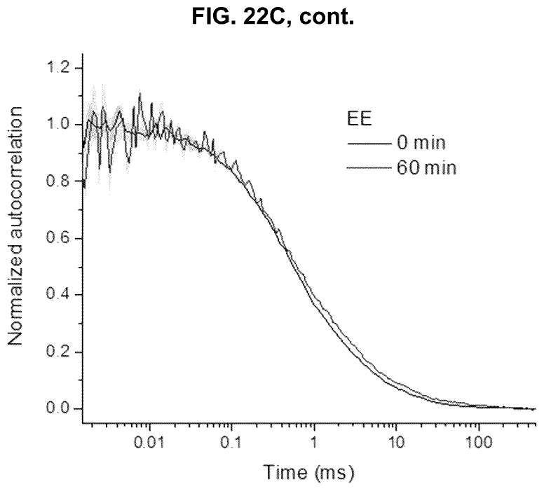

By analogy with NPs, where a protein corona is quickly formed within 1 h upon injection to biological media, it was hypothesized that DNs would follow a similar pattern. Thus, to confirm the protein corona formation, protein adsorption to the surface of DNs was analyzed. All three types of DN were incubated either in serum-containing medium or serum-free buffer for 2 h. After incubation, DNs were collected by centrifugation, washed with PBS buffer, and subjected to 1D sodium dodecyl sulfate polyacrylamide gel electrophoresis (SDS-PAGE) and Coomassie staining for the total proteins detached from the structures (FIG. 22A). Indeed, DNs incubated in HBSS did not carry any proteins (FIG. 22A). By contrast, clear protein bands were eluted from DNs incubated in serum-containing medium (FIG. 22A). In fact, SDS-PAGE and Coomassie staining require denaturing conditions for the analysis. Further, confirmation and monitoring of protein adsorption while the DNs are immersed in the solution were performed utilizing in situ methodology. Thus, fluorescence correlation spectroscopy (FCS) was used, a widely used method enabling precise measurements of the increase in hydrodynamic radius of the particle upon corona formation. The increasing particle size resulting from protein adsorption was assessed by measuring the increase in diffusion time. In fact, a 60 min incubation of DNs in serum-containing medium led to a statistically significant increase of the diffusion time, reflected by a shift of the autocorrelation curve (FIG. 22B-C). Of note, the diffusion time reflects the size of particles; the longer the time the larger the particles, but the hydrodynamic radius calculated from FCS measurements can only be taken as an estimate and its value depends on the chosen assumptions. This is why FIG. 22B shows less processed results in the form of mean diffusion times. If the sizes are estimated on the basis of the calibration measurements and assumption of ideally spherical particles, the diameter of all DNs is 15-28 nm, while the thickness of the protein corona is 2.2±0.8, 1.7±1.1, and 2.1±1.4 nm, for K10, 6HB, and EE, respectively. DNs that were incubated in HBSS buffer showed no signs of the increase of diffusion time and subsequently the size of particles (FIG. 22B-C). Thus, FCS data confirmed the results from Coomassie staining (FIG. 22A) and imply that, upon incubation of DNs in serum-containing medium, a protein corona is formed.

In summary, a comparative investigation analyzed the cellular uptake of differently functionalized DNs in distinct but closely related human hepatic cancer cell lines. The three cell types examined (Alexander, HepG2, and Huh7) showed different internalization efficiency. Overall, this study reveals that the extent of DN internalization and the kinetics of uptake may grossly differ between distinct cell lines, even between phenotypically related cells. The analysis clearly shows that the efficiency of DN engulfment by cells is strongly associated with the cell size. Additionally, modifying DNs with a dense coating of the peptide aurein 1.2 can facilitate endolysosomal escape, which has been a key challenge for the application of DNs in cell delivery studies. DNs rapidly form a protein corona when exposed to serum-containing medium and that this protein corona dramatically reduces the endolysosomal escape efficiency of aurein 1.2-decorated DNs. These results provide a foundation and design strategies for the rational optimization of DN-based delivery vehicles.

It will be apparent to one of ordinary skill in the relevant art that suitable modifications and adaptations to the compositions, formulations, methods, processes, apparata, assemblies, and applications described herein can be made without departing from the scope of any embodiments or aspects thereof. The compositions, apparata, assemblies, and methods provided are exemplary and are not intended to limit the scope of any of the disclosed embodiments. All the various embodiments, aspects, and options disclosed herein can be combined in any variations or iterations. The scope of the compositions, formulations, methods, apparata, assemblies, and processes described herein include all actual or potential combinations of embodiments, aspects, options, examples, and preferences described herein. The compositions, formulations, apparata, assemblies, or methods described herein may omit any component or step, substitute any component or step disclosed herein, or include any component or step disclosed elsewhere herein. The ratios of the mass of any component of any of the compositions or formulations disclosed herein to the mass of any other component in the formulation or to the total mass of the other components in the formulation are hereby disclosed as if they were expressly disclosed. Should the meaning of any terms in any of the patents or publications incorporated by reference conflict with the meaning of the terms used in this disclosure, the meanings of the terms or phrases in this disclosure are controlling. All patents and publications cited herein are incorporated by reference herein for the specific teachings thereof.

Various embodiments and aspects of the inventions described herein are summarized by the following clauses:
Clause 1. A nanoparticle composition comprising a DNA nanostructure (DN) functionalized with an endolysosomal escape peptide.
Clause 2. The composition of clause 1, wherein the DN comprises a 6-helix bundle (6HB) nanostructure.
Clause 3. The composition of clause 2, wherein the 6HB nanostructure comprises six different double-stranded DNA helices, each DNA helix having a nucleotide sequence having at least 90-99% identity to SEQ ID NO: 1-6.
Clause 4. The composition of clause 2, wherein the 6HB nanostructure comprises six different double-stranded DNA helices, each DNA helix having a nucleotide sequence selected from SEQ ID NO: 1-6.
Clause 5. The composition of clause 2, wherein the 6HB nanostructure is a rigid and monomeric assembly roughly 7×6 nm² in size.
Clause 6. The composition of clause 1, wherein the endolysosomal escape peptide coating comprises one or more endolysosomal escape peptides having an amino acid sequence having at least 90-95% identity to SEQ ID NO: 7-12.
Clause 7. The composition of clause 1, wherein the endolysosomal escape peptide coating comprises one or more endolysosomal escape peptides having an amino acid sequence of SEQ ID NO: 7-12.
Clause 8. The composition of clause 7, wherein the endolysosomal escape peptide comprises a lysine10 (K10) peptide (SEQ ID NO: 7).
Clause 9. The composition of clause 7, wherein the endolysosomal escape peptide comprises an aurein 1.2 peptide (SEQ ID NO: 12).
Clause 10. The composition of clause 7, wherein the endolysosomal escape peptide comprises a lysine10 (K10) peptide flanked by two copies of an aurein 1.2 peptide (SEQ ID NO: 10).
Clause 11. The composition of clause 10, wherein the number of copies of the aurein 1.2 peptide per DN is equal to about 40 to about 50.
Clause 12. The composition of clause 1, wherein the diameter of the functionalized DN is from about 15 nm to about 28 nm.
Clause 13. The composition of clause 1, wherein the endolysosomal escape peptide coating binds the DN through electrostatic interactions at a nitrogen/phosphate ratio of about 0.8 to about 1.5.
Clause 14. The composition of clause 13, wherein the nitrogen/phosphate ratio is about 1.
Clause 15. The composition of clause 1, wherein the composition is stable in intracellular lysosomal compartments for up to 24 hr of incubation.
Clause 16. The composition of clause 1, further comprising a therapeutic agent.
Clause 17. A method of improving cellular uptake of a DNA nanostructure (DN) through enhanced endolysosomal escape, the method comprising delivering to a cell a nanoparticle composition comprising a DN functionalized with an endolysosomal escape peptide coating.
Clause 18. The method of clause 17, wherein endolysosomal escape efficiency is determined by a protein corona.
Clause 19. The method of clause 17, wherein cellular uptake efficiency of the functionalized DN is linearly dependent on the cell size.
Clause 20. The method of clause 17, wherein the cell is a hepatoblastoma cell or a hepatocellular carcinoma cell.
Clause 21. The method of clause 17, wherein the endolysosomal escape peptide coating facilitates enhanced endolysosomal escape without concomitant disruption of a cell membrane and without cytotoxicity to the cell.
Clause 22. The method of clause 17, wherein the composition further comprises a therapeutic agent and the method is used to deliver the therapeutic agent to a cell.

### EXAMPLES

### Materials

The following fluorescent probes were used. To visualize the plasma membrane in confocal imaging, the following plasma membrane stains were used: CellMask Green (Cat. No. C37608, Thermo Fisher Scientific), CellMask Orange (Cat. No. C10045, Thermo Fisher Scientific), or CellBrite Blue (Cat. No. 30024, Biotium). Nuclei were counterstained with Hoechst 33342 (Cat. No. H3570, Thermo Fisher Scientific). Lysosomes were labeled with lysosomal marker LysoTracker Blue DND-22 (Cat. No. L7525, Thermo Fisher Scientific). The optimal incubation time for each probe was determined experimentally.

### Fabrication and Characterization of DNs

All oligonucleotides were obtained from Integrated DNA Technologies (Coralville, Iowa) and purified using 14% urea-based denaturing polyacrylamide gel electrophoresis (PAGE). One strand was labeled with AlexaFluor-488 for imaging in the agarose gels and in microscopy experiments. Each strand was added to a mixture at 10 UM in 1× Tris-acetic acid-EDTA (TAE) buffer with 12.5 mM MgCl₂ and annealed from 95 to 4° C. over 2 h. The successful formation of the 6-helix bundle was confirmed using agarose gel electrophoresis. DN size was characterized utilizing a Zetasizer Nano (Malvern Instruments). DNs were dispersed in PBS, pH 7.4.

### Atomic Force Microscopy

AFM images were captured on a Bruker Multimode 8 system with Nanoscope V controller in a ScanAsyst in Fluid mode with ScanAsyst-Fluid+ AFM probes (Bruker, k~0.7 N/m, tip radius<10 nm). Two microliters of sample were deposited on freshly cleaved mica followed by the addition of 48 μL of 1×TAE with 12.5 mM Mg²⁺ for 2 min. One mM NiCl₂ buffer can be used to enhance the adsorption of DNA nanostructures on the mica surface.

DNs Stability Assay with Melting Profile Analysis

The melting transitions of the DNA nanostructures were assessed using previously published methodology utilizing a MX3005P real-time thermocycler (Stratagene). The DNs were assembled containing FRET reporter dyes [6-carboxyfluorescein (FAM) donor and TAMRA acceptor] pairs (folded at 1 μM in 1×TAE with 12.5 mM Mg²⁺). The DNA constructs were diluted into the stated buffer systems to give a final DNA concentration of 0.15 μM (total volume of 300 μL) in eight-well optical tube strips (Agilent, 100 μL per tube). Optical quality sealing tape (Agilent) was placed on top to prevent evaporation. The samples were heated from 25 to 80° C. at a rate of 0.5° C. per min. The efficiency of energy transfer (E) was determined at each temperature according to E (T)=1−I_{DA}(T)/I_{D}(7), where I_{DA} and I_{D} are, respectively, the fluorescence intensities of the FRET donor (FAM) in the presence and absence of the FRET acceptor (TAMRA). All experiments were repeated in three replicates to ensure reproducibility. The melting temperature was determined from taking the first derivative of the donor fluorescence profile.

### Peptide Synthesis and Characterization

All peptides were synthesized on a CEM Liberty Blue using a Rink amide resin via standard Fmoc-based solid phase peptide synthesis. Briefly, 0.5 M diisopropylcarbodiimide was used as an activator, 1 M oxyma with 0.1 M diisopropylethylamine was used as an activator base, and 20% piperidine was used as a deprotecting agent. The peptide was cleaved from the resin using a solution of 95% trifluoroacetic acid with 2.5% triisopropylsilane and 2.5% water, followed by ether precipitation. Following pellet suspension, the crude peptide was purified on a reverse phase HPLC instrument (Waters), using a gradient of 0-100% acetonitrile with 0.1% TFA. Pure fractions were identified using MALDI-TOF mass spectrometry (Bruker Microflex) with α-cyano-4-hydroxycinnamic acid as a matrix. The K10-cysteine was labeled with a maleimide-C2-pHrodo Red dye by addition of the dye (10 equiv) in PBS pH 7.

### DN Coating and Characterization

The DNs (1 μM) were mixed with the desired K10-containing peptide at a 1:1 N/P ratio and incubated at room temperature for a minimum of 2 h. All coated DNs used for cell experiments utilized the pHrodo-labeled K10 at 20 mol % of the total K10 concentration. All coated DNs run on agarose gels utilized the fluorescein labeled K10 at 20 mol % of the total K10 concentration. In order to determine the optimal N/P ratio for complete coating of the DNs, the structures were electrophoresed using 1.5% agarose gels at 65 V for 60 min and imaged using the fluorescein labeled K10.

### Cell Culture

In this study, established cellular models of hepatic cells were utilized, namely, the human hepatoblastoma HepG2 cell line (American Type Culture Collection, ATCC) and the human hepatocellular carcinoma cell lines Alexander (PLC/PRF/5, ATCC) and Huh7 (Japanese Collection of Research Bioresources, JCRB). Standard culturing media composition was used, i.e., EMEM medium (ATCC) supplemented with 10% fetal bovine serum (FBS, Thermo Fisher Scientific) and 1% penicillin/streptomycin (Thermo Fisher Scientific). Mycoplasma testing, using the MycoAlert mycoplasma detection kit (LT07-418, Lonza, Basel, Switzerland), was performed routinely. Cells were grown in a humidified 5% CO₂ atmosphere at 37° C. Once per week, fresh cell culture medium was added.

### Cell Viability Assay

The potential toxicity of synthesized DNs was assessed using a well-established alamarBlue viability assay (Thermo Fisher Scientific). The technique is based on the cleavage of resazurin to resorufin by undamaged live cells. This cleavage leads to an increase of the overall alamarBlue color intensity. Subsequently, the percentage of metabolically active cells in the culture was calculated on the basis of the absorbance. Cell viability was assessed via the alamarBlue assay according to guidelines of the manufacturer and previously established treatment protocol. In short, distinct cell lines were grown in 96-well plates at a density of 10,000 cells per well and incubated with different concentrations of DNs for 24 h. Afterward, the alamarBlue reagent was supplemented to each well, and plates were incubated for 2 h at 37° C. The TECAN microplate reader SpectraFluor Plus (TECAN, Mannedorf, Switzerland) was utilized to detect the absorbance of the alamarBlue reagent at 570 nm. Readings were done in triplicate, with three independent experiments performed for each measurement. Furthermore, DN interference was analyzed with the assay reagent and verified that the nanostructures do not react with alamarBlue (data not shown).

### Cellular Uptake Analysis

Confocal microscopy was utilized to assess the cellular uptake of DNs. To analyze intracellular DN distribution, cells were cultured in 6-channel μ-Slides (Ibidi, Martinsried) and treated with different concentrations of fluorescently labeled DNs for 1, 6, and 24 h. Then, cells were fixed with 4% paraformaldehyde (VWR) and stained with CellBrite Blue (Biotium) plasma membrane stain. Labeled cells were visualized using spinning disk confocal microscopy IXplore SpinSR (Olympus, Tokyo, Japan) according to verified protocols. For live cell imaging, the cell membrane was labeled with a CellMask Orange (Thermo Fisher Scientific) plasma membrane stain. Dual-color imaging of confocal cross sections was performed at about half the cell height for the quantitative assessment of DN intracellular distribution. From the image of the stained membrane, binary masks were extracted that enabled the definition of the membrane-associated regions and the cytosolic space. The corresponding image of the cell membrane was converted into a mask of the cell in all imaged confocal planes. By applying this mask to the relevant image of DNs, the engulfed particles can be discriminated. The intracellular DNs were measured as the corrected total cell fluorescence (CTCF) of the full area of interest, i.e., intracellular region bordered by cell membrane mask. A published methodology was used to define the net average CTCF intensity for each image. The CTCF was calculated by following formula: CTCF=integrated density−(area of selected cell×mean fluorescence of background readings). The mean fluorescence of the background was defined as an image area without fluorescent objects. CTCF was determined as the sum of pixel intensity for a single image with the subtracted average signal per pixel for a region selected as the background, according to previously published methodology. Image quantifications were performed using ImageJ software (NIH).

### Analysis of DN Stability in Cells by FRET Imaging

An Olympus confocal imaging system (Olympus, Tokyo, Japan), described below, was used for FRET measurements. Cells were grown in 6-channel μ-Slides (Ibidi, Martinsried) and incubated with 6HB-containing FRET reporter dyes [6-carboxyfluorescein (FAM) donor and TAMRA acceptor] at a 50 nM concentration for 24 h. For imaging, the FAM cells were excited with the 488 nm laser and fluorescence was collected with a BA510-550 filter (Olympus), whereas the FRET-signal was detected with a BA575IF filter (Olympus). To image TAMRA, the 561 nm excitation laser was utilized while emission was detected using a BA575IF filter (Olympus). Confocal FRET analysis was performed as described in the “FRET and colocalization analyzer-Users Guide.” 6HBs containing either 6-carboxyfluorescein (FAM) donor or TAMRA acceptor only were used as negative controls.

### DN-Protein Interaction

DNs at 50 nM concentration were incubated either in HBSS, or in EMEM medium (ATCC) supplemented with 10% fetal bovine serum (FBS, Thermo Fisher Scientific) for 2 h at 37° C. Centrifugation was utilized to collect the particles. To remove any remaining unbound proteins, DNs were washed extensively with PBS. The samples were centrifuged for 15 min at 15,000×g followed by pellet resuspension in PBS. The washing with PBS was performed three times to eliminate all the molecules not bound to DNs. Such methodology has been shown to be effective for the isolation of particle-protein corona complexes. Indeed, the main aim of this work was not the most accurate possible determination of the protein corona composition but rather the demonstration that the formation of protein corona occurs. The process of elution and denaturation in sample loading buffer was used to detach proteins associated with the particles. Afterward, proteins were separated by gel electrophoresis (1D SDS-PAGE). Full cell culture EMEM medium supplemented with 10% fetal bovine serum was utilized as a control. Gels were stained with Coomassie blue (AppliChem).

### Protein Corona Analysis Using Fluorescence Correlation Spectroscopy

To test for the formation of protein corona on the particles, their diffusivity was measured using fluorescence correlation spectroscopy (FCS). The method is based on the analysis of the fluorescence intensity fluctuations resulting from a diffusion of diluted fluorescent particles through a small volume (~1 fL) from which the signal is collected. The signal is autocorrelated and fitted to a 3D free diffusion model to get the mean diffusion time, TD. Under the assumption of reasonably unchanged shape of the particles, this parameter is proportional to the hydrodynamic radius of the particles according to the Stokes-Einstein equation. Therefore, the increase of TD, can be interpreted as an enlargement of the particles, e.g., due to protein corona formation. FCS data acquisition was carried out by utilizing an inverted confocal fluorescence microscope, Olympus IX71 (Olympus, Hamburg, Germany), equipped with single-photon counting unit MicroTime 200 (PicoQuant, Berlin, Germany). An excitation of 470 nm was achieved with a diode laser (LDH-μ-C-470; PicoQuant, Berlin, Germany) operating at 20 MHz. A water immersion objective (1.2 NA, 60×, Olympus) was utilized to visualize a sample. The fluorescence signal was collected through the main dichroic mirror (Z473/635, Chroma, Rockingham, VT), a 50 μm pinhole, and guided to the single photon avalanche diode using 515/50 band-pass filter (Chroma). All FCS data acquisitions were carried out at 25° C. in 8-well μ-Slides (Ibidi, Gra\ddot{f}elfing, Germany). Due to particle adsorption to the glass, plastic bottom μ-Slides were used, which showed resistance to adsorption. Atto 488 (Atto-tec, Siegen, Germany) dye was used as a reference for calibration measurements.

The measured data were fitted utilizing a standard 3D diffusion model implemented in Symphotime 64 software (PicoQuant, Berlin, Germany). Fluorescence decay data were used to correct for the noise. On the basis of the intensity histogram, a small fraction of particles with the highest intensity (>99.8% threshold) was excluded from the analysis as possible aggregates. Mean diffusion times obtained from the fitting of the data from three separate experiments were used to calculate the average diffusion time; the weighted-average based on the error of the fitting was used.

### High-Resolution Spinning Disk Confocal Microscopy

In order to be able to reveal clear subcellular details of DNs localization, a novel IXplore SpinSR Olympus high-resolution imaging system (Olympus, Tokyo, Japan) was used.

Additionally, 6-channel μ-Slides (Ibidi, Martinsried) were utilized for cell seeding. Afterward, cells were treated with different concentrations of fluorescently labeled DNs. Then cells were stained for CellBrite Blue or LysoTracker Blue DND-22. The imaging system consists of the following units: an inverted microscope (IX83; Olympus, Tokyo, Japan) and a spinning disc confocal unit (CSUW1-T2S SD; Yokogawa, Musashino, Japan). Fluorescence data for image reconstruction were collected via either a 100× silicone immersion objective (UPLSAPO100×S NA 1.35 WD 0.2 silicone lens, Olympus, Tokyo, Japan) or a 20× objective (LUCPLFLN20×PH NA 0.45 air lens, Olympus, Tokyo, Japan). The following lasers were used to excite fluorophores: 405 nm laser diode (50 mW), 488 nm laser diode (100 mW), and 561 nm laser diode (100 mW). Confocal images were acquired at a 2048×2048-pixel resolution. The fluorescent images were collected by appropriate emission filters (BA420-460; BA575IF; BA510-550; Olympus, Tokyo, Japan) and captured concurrently by two digital CMOS cameras ORCA-Flash4.0 V3 (Hamamatsu, Hamamatsu City, Japan). Fluorescence confocal images were acquired using software cellSens (Olympus, Tokyo, Japan). Quantitative image analysis was performed by selecting randomly ~5-10 visual fields per each sample, using the same setting parameters (i.e., spinning disk speed, laser power, and offset gain). ImageJ software (NIH) was used for image processing, quantification, and 3D reconstruction.

### Image Quantification

To measure cell size, cells were stained with CellMask Green (Thermo Fisher Scientific) plasma membrane stain. Nuclei were counterstained with Hoechst 33342 (Thermo Fisher Scientific). Spinning disk confocal microscopy IXplore SpinSR (Olympus, Tokyo, Japan) was used to acquire images of the labeled cells. The analysis of DN uptake is described above.

To analyze the endolysosomal escape of DNs colocalization analysis was performed. Cells were incubated with different types of DNs (at 50 nM concentration) for 6 h either in full medium (10% FBS EMEM) or in serum-free medium (0% FBS EMEM). After incubation, cells were labeled with lysosomal marker LysoTracker Blue DND-22 (Thermo Fisher Scientific). Stained cells were analyzed using the confocal system described above. Fluorescence images were acquired by software cellSens (Olympus, Tokyo, Japan). In order to quantitatively assess colocalization, the Pearson correlation coefficient was calculated. To robustly analyze overall association between two fluorescent probes, it is well-established to calculate the Pearson correlation coefficient, which defines pixel-by-pixel correlation by representing mean-normalized to values from −1 (anticorrelation) to +1 (correlation). The Pearson correlation coefficient for fluorophore pairs (either DNA-lysosomes or DNA-pHrodo) was calculated using the Coloc 2 tool available in ImageJ.120

### Statistical Analysis

Cellular viability was analyzed and represented as mean±SEM. The ANOVA analysis with subsequent Newman-Keuls test was utilized to assess the statistical significance of differences between the groups. MaxStat Pro 3.6 software (MaxStat Software, Cleverns, Germany) was used to perform all statistical analyses. Differences were considered statistically significant at (*) P<0.05. Correlation analysis between the cell size and cellular uptake of DNs was done utilizing Spearman rank order correlation. Correlation coefficients and P values were calculated using SigmaPlot 13 software (Systat Software, Inc.).

Fluorescence microscopy analysis (namely, the analysis of cell size and uptake and colocalization of DNA-lysosomes or DNA-pHrodo) was subjected to quantitative assessment in accordance with rigorously defined guidelines. Guidance for quantitative confocal microscopy was employed to perform a quantitative assessment in accordance with previous publications. Quantitative microscopy analysis was carried out using images from three independent experiments. Each microscopy experiment included 10 randomly selected fields from each sample. The determination of sample size was performed in accordance with a previously published statistical methodology. Accordingly, the sample size for 95% confidence level and 0.9 statistical power was calculated as n=30. Therefore, at least 30 randomly selected cells were analyzed for statistically relevant fluorescence microscopy image quantification.

Overall, a statistical methodology was used to determine the sample size, assuming 95% confidence level and 0.9 statistical power.

What is claimed:

1. A nanoparticle composition comprising a DNA nanostructure (DN) functionalized with an endolysosomal escape peptide having at least 95-99% identity to SEQ ID NO: 10.

2. The composition of claim 1, wherein the endolysosomal escape peptide is SEQ ID NO: 10.

3. The composition of claim 1, wherein the DN comprises a 6-helix bundle (6HB) nanostructure.

4. The composition of claim 3, wherein the 6HB nanostructure comprises six double-stranded DNA helices formed by complementary nucleotides of SEQ ID NO: 6 and SEQ ID: NO: 5; SEQ ID NO: 5 and SEQ ID NO: 4; SEQ ID NO: 4 and SEQ ID: NO: 3; SEQ ID NO: 3 and SEQ ID NO: 2; SEQ ID NO: 2 and SEQ ID: NO: 1; and SEQ ID NO: 1 and SEQ ID NO: 6.

5. The composition of claim 4, wherein the 6HB nanostructure is a rigid and monomeric assembly of about 7×6 nm² in size.

6. The composition of claim 1, wherein the number of copies of endolysosomal escape peptide per DN is equal to about 40 to about 50.

7. The composition of claim 1, wherein the diameter of the functionalized DN is from about 15 nm to about 28 nm.

8. The composition of claim 1, wherein the endolysosomal escape peptide binds the DN through electrostatic interactions at a nitrogen/phosphate ratio of about 0.8 to about 1.5.

9. The composition of claim 8, wherein the nitrogen/phosphate ratio is about 1.

10. The composition of claim 1, wherein the composition is stable in intracellular lysosomal compartments for up to 24 hr of incubation.

11. The composition of claim 1, further comprising a therapeutic agent.

12. A method of improving cellular uptake of a DNA nanostructure (DN) through enhanced endolysosomal escape, the method comprising delivering to a cell a nanoparticle composition comprising a DN functionalized with an endolysosomal escape peptide having at least 95-99% identity to SEQ ID NO: 10.

13. The method of claim 12, wherein endolysosomal escape efficiency is determined by a protein corona.

14. The method of claim 12, wherein cellular uptake efficiency of the functionalized DN is linearly dependent on the cell size.

15. The method of claim 12, wherein the cell is a hepatoblastoma cell or a hepatocellular carcinoma cell.

16. The method of claim 12, wherein the endolysosomal escape peptide facilitates enhanced endolysosomal escape without concomitant disruption of a cell membrane and without cytotoxicity to the cell.

17. The method of claim 12, wherein the composition further comprises a therapeutic agent and the method is used to deliver the therapeutic agent to the cell.
